(19) United States
(12) Patent Application Publication
(10) Pub. No.: US 2025/0360099 A2
(43) Pub. Date: Nov. 27, 2025

(54) A significantly non-toxic novel Cobalt(III) Schiff base complex induces apoptosis via G2-M cell cycle arrest in human breast cancer cell line MCF-7 and cell cycle arrest of colon cancer cell lines HCT-116 and SW- 480 via G0-G1

(71) Applicant: CHITTARANJAN NATIONAL CANCER INSTITUTE, Kolkata, West Bengal (IN)

(72) Inventors: Sanchari Dasgupta, Versailles Cedex (FR); Kanisha PAL Kar, Kolkata, West Bengal (IN); Atish Barua, Boston, MA (US); Diya Ghosh, Kolkata, West Bengal (IN); Bikash Kabi, Kolkata, West Bengal (IN); Koushik Dewan, Kolkata, West Bengal (IN); Arpita Chandra, Kolkata, West Bengal (IN); Sunandita Bhar, Kolkata, West Bengal (IN); Tanima Das, Kolkata, West Bengal (IN)

(21) Appl. No.: 18/827,331

(22) Filed: Sep. 6, 2024

Related U.S. Application Data
(63) Continuation-in-part of application No. 20/233,106, filed on Sep. 8, 2023.

(30) Foreign Application Priority Data
Sep. 8, 2023 (IN) 202331060593

(51) Int. Cl. A61K 31/295; A61P 35/00; C07F 15/06

(52) U.S. Cl. CPC A61K 31/295; A61P 35/00; C07F 15/065

(57) ABSTRACT

A significantly non-toxic novel mononuclear cobalt(III)-Schiff base complex (1) capable to induce apoptosis via G2-M cell cycle arrest in human breast cancer cell line MCF-7 and cell cycle arrest of colon cancer cell lines HCT-116 and SW-480 via G0-G1. The Schiff base complex (1) having >99% purity has been synthesized by a facile “One pot” synthesis method and has been characterized with standard spectroscopic techniques. Complex 1 exhibits cytotoxicity (IC₅₀=16.81±1.33 μM) at much lower concentration in comparison to oxaliplatin (IC₅₀=31.4±0.69 μM) against MCF-7 cells and causes apoptosis in colon cancer cell lines HCT-116 and SW-480 by arresting the cell cycle at the G0-G1 phase having IC₅₀ values of 15.27±1.18 μM and 10.04±1.98 μM respectively comparable with the IC₅₀ values of oxaliplatin which are 16.73±1.78 μM and 7.87±1.54 μM respectively after 24 h of treatment without being overly toxic to human PBMCs (IC₅₀=>60 μM). In vivo subacute toxicity (28 days) and systemic chronic toxicity (40 days) studies were carried out in normal Swiss albino mice showed 1 is significantly nontoxic to the host.

### CROSS REFERENCE TO RELATED APPLICATION(S)

This application is based on and hereby claims the benefit under 35 U.S.C. § 119 from Indian Patent Application No. 202331060593 filed on Sep. 8, 2023. This application is a continuation-in-part of Indian Patent Application No. 202331060593, the contents of which are incorporated herein by reference.

### FIELD OF INVENTION

Present invention is about Metal complexes relating to the field of chemotherapy for newer, more effective but lesser toxic anticancer agents. More particularly the present invention relates to a cobalt (III)-Schiff base complex exhibiting cytotoxicity at lower concentration in comparison to oxaliplatin against cancerous cells for 24 h of therapy without being overly toxic to the normal healthy human cell line (PMBC).

### BACKGROUND OF INVENTION

Despite significant improvements in biomedical research, female breast cancer continues to be the major public health concern as the leading cause of cancer death amongst women globally [Lei, S. et al., Global patterns of breast cancer incidence and mortality: A population-based cancer registry data analysis from 2000 to 2020, Cancer Commun. 41 (2021) 1183-1194]. These findings pointing toward the fact that further improvement in the therapeutic field is needed. Since ancient times various metal-based therapies are used in the field of medicine due to their widespread biological and pharmaceutical properties [Mjos, K. D. et al., Metallodrugs in Medicinal Inorganic Chemistry, Chem. Rev. 114 2014; Hambley, T. W.: Developing new metal-based therapeutics: challenges and opportunities, Dalton Trans. (2007) 4929-4937; Budzisz, E.: Role of Metal Ions Complexes and their Ligands in Medicine, Pharmacy and Cosmetology, Curr. Med. Chem. 26 (2019) 578-579]. The discovery of cisplatin about 58 years ago ushered in a new age of cancer treatment, piquing researchers' interest in inorganic metal-based chemotherapeutics. Platinum complexes have been in use for the treatment and management of several cancer types including breast cancer over decades [Garufi, C. et al., Single-agent oxaliplatin in pretreated advanced breast cancer patients: a phase II study, Ann. Oncol. Off. J. Eur. Soc. Med. Oncol. 12 (2001) 179-182; Martín, M.: Platinum Compounds in the Treatment of Advanced Breast Cancer, Clin. Breast Cancer. 2 (2001) 190-208]. Despite the proven therapeutic efficacy of platinum complexes, cisplatin and other platinum-based drugs have been associated with nephrotoxicity, ototoxicity, neurotoxicity, poor selectivity towards cancer cells, and a high risk for cancer cells to develop resistance to them [Rosenberg, B. et al., Inhibition of Cell Division in Escherichia coli by Electrolysis Products from a Platinum Electrode, Nature. 205 (1965) 698-699; Dilruba, S. et al.; Platinum-based drugs: past, present and future, Cancer Chemother. Pharmacol. 77 (2016) 1103-1124]. Oxaliplatin, an analogue of cisplatin and a third-generation chemotherapeutics, having the 1,2-diaminocyclohexane ligand can overcome acquired drug resistance and toxicity of cisplatin to some extent [Siddik, Z. H. et al., Antitumor activity of isomeric 1,2-diaminocyclohexane platinum (IV) complexes, J. Cancer Res. Clin. Oncol. 120 (1994) 409-414]. However US patent application number 719689 9/02/76 disclosed Malonato-, Hydroxymalonato-, Dinitro-, Hydroxonitrato- & Sulfato(1,2-Diaminocyclohexane) Platinum(II), prepared from dichloro(1,2-diaminocyclohexane were more effective than dichloro (1,2-diaminocyclohexane)platinum(II) in the treatment of L210 leukemia in mice, both alone & in combination with Cyclophosphamide or Yoshi 864 [PubChem, 1,2-Cyclohexanediamine, (n.d.). https://pubchem.ncbi.nlm.nih.gov/compound/4610 (accessed Jul. 14, 2021).

Although oxaliplatin is effective in the treatment of metastatic colon cancer, breast cancer, cervical cancer, and non-small cell lung cancer [Raez, L. E. et al., Oxaliplatin in First-line Therapy for Advanced Non-Small-Cell Lung Cancer, Clin. Lung Cancer. 11 (2010) 18-24; Ban, H et al., Efficacy and safety of docetaxel plus oxaliplatin as a first-line chemotherapy in patients with advanced or metastatic non-small cell lung cancer: Docetaxel and oxaliplatin for NSCLC, Thorac. Cancer. 5 (2014) 525-529] but it causes hepatotoxicity and nephrotoxicity [Rubbia-Brandt, L. et al., Severe hepatic sinusoidal obstruction associated with oxaliplatin-based chemotherapy in patients with metastatic colorectal cancer, Ann. Oncol. 15 (2004) 460-466; Wolf, P. S. et al., Preoperative Chemotherapy and the Risk of Hepatotoxicity and Morbidity after Liver Resection for Metastatic Colorectal Cancer: A Single Institution Experience, J. Am. Coll. Surg. 216 (2013) 41-49.; Jagieła, J. et al., Nephrotoxicity as a Complication of Chemotherapy and Immunotherapy in the Treatment of Colorectal Cancer, Melanoma and Non-Small Cell Lung Cancer, Int. J. Mol. Sci. 22 (2021) 4618].

Researchers have therefore paid attention to develop new transition metal complexes with reduced toxicity and increased efficacy. To achieve this purpose, several metal complexes have been synthesized with different ligands and metal ions, and their anticancer activity has been explored in vitro and in vivo. A series of mononuclear complexes with Co(II), Ni(II), Cu(II), Zn(II), Hg(II), Mo(VI) and Pd(II) containing the Schiff base ligand derived from the 1:2 condensation of 2,6-diformyl-4-methylphenol and 5,6-diamino-1,3-dimethyluracil were synthesized. The complexes were evaluated for antiproliferative behaviour against five human tumor cell lines (human neuroblastoma NB69, human breast cancer MCF-7 and EVSA-T, human glioma H4 and human bladder carcinoma cell line ECV) suggested a modulator behaviour, according to the concentration, of cell growth due to their estrogen-like characteristics [Illán-Cabeza, N. A. et al., Synthesis, characterization and antiproliferative activity of metal complexes with the Schiff base derived from the condensation 1:2 of 2,6-diformyl-4-methylphenol and 5,6-diamino-1,3-dimethyluracil, J. Inorg. Biochem. 102 (2008) 647-655].

The necessary trace clement cobalt plays many important physiological roles in human biological systems, including DNA synthesis, formation of red blood cells, and maintaining the health of nerves. This indicates that the human body system can tolerate the excess cobalt overburden. The cobalt ions are also involved in oxidative stress-induced apoptosis mediated by mitochondria in cancerous cells [V. Battaglia, et al., Cobalt induces oxidative stress in isolated liver mitochondria responsible for permeability transition and intrinsic apoptosis in hepatocyte primary cultures, Int. J. Biochem. Cell Biol. 41 (2009) 586-594]. This participation of cobalt ions and the interaction of cobalt compounds with DNA is a key feature of their cytotoxicity, which supports their potential as good chemotherapeutics.

It is interesting to note that cobalt has a high affinity for cysteines and histidine of zinc finger proteins, that are associated with cancer progression. A cobalt(III) Schiff base with modified oligonucleotide has been proved as valuable cancer therapeutics for selectively targeting zinc finger protein in vitro. Following the success story of cobalt, a number of Co(II) and Co(III) Schiff base complexes have been synthesized and studied as an anticancer agents [Harney, A. S. et al., Targeted inhibition of Snail family zinc finger transcription factors by oligonucleotide-Co(III) Schiff base conjugate, Proc. Natl. Acad. Sci. 106 (2009) 13667-13672; Gowdhami, B. et al., Potential application of two cobalt (III) Schiff base complexes in cancer chemotherapy: Leads from a study using breast and lung cancer cells, Toxicol. In Vitro. (2021) 105201. https://doi.org/10.1016/j.tiv.2021.105201]. The above teaches that Cobalt-containing complexes are one such class of metal complexes, that could be effective as alternative metal-based chemotherapeutics instead of platinum compounds [Donaldson, J. D. et al., Cobalt and Cobalt Compounds, in: Ullmanns Encycl. Ind. Chem., American Cancer Society, 2005.; Heffern, M. C. et al., Cobalt derivatives as promising therapeutic agents, Curr. Opin. Chem. Biol. 17 (2013) 189-196.].

Literature survey unveils that most of the mononuclear cobalt Schiff base complexes are effective against breast or lung cancer cells, and their mechanism of action is similar to that of platinum complexes, promoting DNA cleavage and thus producing ROS, arresting cell cycle in the G2-M phase, which leads to apoptosis via a mitochondria-mediated intrinsic pathway. In particular, the majority of Cobalt Schiff base complexes exhibit strong anticancer activity against human breast cancer cell line MCF-7 [Kar, K.; Ghosh, D.; Kabi, B.; Chandra, A.: A concise review on cobalt Schiff base complexes as anticancer agents, Polyhedron. 222 (2022) 115890]. Human breast cancer cell line MCF-7 is a “Luminal A” subtype comprise the majority of breast cancers, accounting for at least 75% of all cases. So, testing these types of cobalt complexes against MCF-7 cell line could be very rational strategy from this point of view. Although few reported Cobalt Schiff base complexes exhibit cytotoxicity against cancer cells and may act as a good alternative to cisplatin but detailed toxicity profiling is needed to project them as future chemotherapeutics.

The Schiff base complexes are particularly interesting for their facile synthesis, metal-chelating properties, and flexibility with which they can regulate biochemical action [Kumar, S. et al.: Applications of metal complexes of Schiff bases-A review, 68 (2009) 7]. Despite the extensive versatility of Cobalt Schiff base derivatives, only one compound, Doxovir has reached phase-II clinical trials. It is effective against drug-resistant herpes simplex virus, though its detailed molecular mechanism is still unknown [Schwartz, J. A. et al., Herpes simplex virus type 1 entry is inhibited by the cobalt chelate complex CTC-96, J. Virol. 75 (2001) 4117-4128], Thus there is an urgent need for efficient anticancer agent having detailed toxicity profiling exhibiting nominal side effects and toxicity.

### OBJECTIVE OF THE INVENTION

Primary objective of the present invention is to provide Platinum free efficient anticancer agent having nominal side effects and toxicity involving Co metal ion preferably more suitable against human breast cancer or other human cancers including colon cancer

Another preferred objective of the present invention is to provide said efficient anticancer agent based on Schiff base complex of 2,6-diformyl-4-methylphenol and 1,2-diaminocyclohexane and Co metal ion.

Another objective of the present invention is to provide said efficient anticancer agent which will have detailed toxicity profiling exhibiting nominal side effects and toxicity.

Another objective of the present invention is to provide said efficient anticancer agent which will be fluorescent in nature such that after entering into the cells the anticancer agent would fluores well under microscope attaining advantage for easy tracking of the agent.

Another objective of the present invention is to provide said efficient anticancer agent which would be more potent than Oxaliplatin but devoid of nephrotoxicity, ototoxicity, neurotoxicity, hepatotoxicity and poor selectivity towards normal cells

Another objective of the present invention is to provide a process to provide said efficient anticancer agent with >99% purity which will be easy to perform and scale up for commercial production.

### SUMMARY OF THE INVENTION

In the primary embodiment the present invention is directed to provide a non-toxic mononuclear side-off compartmental cobalt (III) Schiff base complex which is reaction product C₂₄H₃₀N₃O₁₀Co selectively of Co(III) containing Schiff base ligand and derived of 1,2-diaminocyclohexane (DACH) and 2,6-diformyl-4-methylphenol (Dif) which is capable of exhibiting cytotoxicity at lower concentration against cancerous cells for 24 h of therapy without being overly toxic to the normal healthy human cell line (PMBC).

A preferred embodiment of the present invention is directed to provide said non-toxic mononuclear side-off compartmental cobalt(III) Schiff base complex which is reaction product C₂₄H₃₀N₃O₁₀Co selectively of Co(III) containing Schiff base ligand and derived of 1,2-diaminocyclohexane (DACH) and 2,6-diformyl-4-methylphenol (Dif) which is capable of causing apoptosis in breast cancer cell line MCF-7 cells by arresting the cell cycle at the G2-M phase at much lower concentrations than oxaliplatin having IC₅₀ values of 16.81±1.33 μM in compare to IC₅₀ value of 31.4±0.69 μM after 24 h treatment against MCF-7 cells without being overly toxic to healthy host systems.

Another preferred embodiment of the present invention is directed to provide said non-toxic mononuclear side-off compartmental cobalt(III) Schiff base complex which is reaction product C₂₄H₃₀N₃O₁₀Co selectively of Co(III) containing Schiff base ligand and derived of 1,2-diaminocyclohexane (DACH) and 2,6-diformyl-4-methylphenol (Dif) which is capable of causing apoptosis in colon cancer cell lines HCT-116 and SW-480 by arresting the cell cycle at the G0-G1 phase having IC₅₀ values 15.27±1.18 μM and 10.04±1.98 μM respectively comparable with the IC₅₀ values of oxaliplatin which are 16.73±1.78 μM and 7.87±1.54 μM respectively for HCT-116 and SW-480 cells after 24 h treatment.

Another embodiment of the present invention is directed to provide said non-toxic mononuclear Schiff base complex have fluorescence property; Displays a broad band consisting of two small breaks at 1634 cm⁻¹ and 1642 cm⁻¹ and sharp bands at ˜1550 cm⁻¹ and ˜1385 cm⁻¹ by FTIR,

shows a strong band at λₘₐₓ˜410 nm in methanol, but two bands at λₘₐₓ˜412 nm and 354 nm in 0.03% DMSO-H₂O solution and stable upto one week in 0.03% DMSO-H₂O medium as indicated by UV-Vis spectroscopy;
retention time of 2.018 min in HPLC run on an C-18, Reserve Phase Column from WATERS (4.6×150 mm, particle size 5 μm), using 0.1% TFA in H₂O and 100% CH₃CN as mobile phase;
retains its mononuclear entity in the solution phase and presence of cobalt ion in the +3 oxidation state is confirmed by ESI-MS and Cyclic Voltammetry study respectively; and fluoresces well under microscope after entering the cells.

Yet another embodiment of the present invention is directed to provide said non-toxic mononuclear Schiff base complex exhibit cytotoxic effects on MCF-7 cells after 24 h of incubation with IC₅₀ values of 16.81±1.33 μM whereas oxaliplatin inhibit the cell viability of MCF-7 cells with an IC₅₀ value of 31.4±0.69 μM after 24 h treatment causing significant reduction of the number of viable MCF-7 cells than that of the untreated group whereas do not impart any significant cytotoxicity in normal PBMCs and results in cell viabilities above 70% under the concentration range tested (5 μM-60 μM) i.e. IC₅₀ of said Schiff base complex for PBMC must be >60 μM.

A further embodiment of the present invention is directed to provide said non-toxic mononuclear Schiff base complex wherein the cobalt(III) Schiff base complex which is reaction product C₂₄H₃₀N₃O₁₀Co synergistically triggers apoptosis and suppressed cell proliferation in MCF-7 cells wherein the levels of pro-apoptotic Bax protein and tumor suppressive protein P53are significantly enhanced, and the levels of antiapoptotic proteins Bcl-2 and Bcl-xL and cell proliferating biomarker PCNA are significantly downregulated and displays the cell migration rate almost similar to oxaliplatin in magnitude after treated with said Schiff base complex at IC₅₀ dose.

Still further embodiment of the present invention is directed to provide said non-toxic mononuclear Schiff base complex wherein cobalt(III) Schiff base complex which is reaction product C₂₄H₃₀N₃O₁₀Co which upon exposure to MCF-7 cells at the IC₅₀ concentration of said Schiff base complex for 24 h showed an increased population of cells in the G2-M phase, 23.1%, compared to 8.88% in the untreated control group; along with reduction of the population of cells in the G0-G1 and S phases indicating cell cycle arrest in the G2-M phase wherein the oxaliplatin treatment increased the population of MCF-7 cells in the G2-M phase of the cell cycle to 21.3% indicating a similar extent of the cell cycle arrest at the G2-M phase like oxaliplatin.

Still further embodiment of the present invention is directed to provide said non-toxic mononuclear Schiff base complex wherein cobalt(III) Schiff base complex which is reaction product C₂₄H₃₀N₃O₁₀Co upon standardized sub-acute systemic toxicity study (28 days) and/or standardized chronic toxicity study (40 days) specified that the animals at different dose groups (from 10 μg/kg of body weight to 40 μg/kg of body weight) do not show any significant changes in the haematological parameters like RBC, WBC, PLT, and HGB as compared to the untreated group of animals implying no significant haematological toxicity and any alteration in the level of biochemical parameters such as SGOT, SGPT, ALP (indicative of drug-induced liver injury), or urea and creatinine (indicative of toxin-induced damaged renal function) expressing hepatotoxicity and nephrotoxicity are not noticed in those biochemical parameters.

Another preferred embodiment of the present invention is directed to provide a process for the preparation of said non-toxic mononuclear Schiff base complex comprises the steps of:
providing ethanolic solution of 2,6-diformyl-4-methylphenol;
providing adding ethanolic solution of (±) cyclohexane-1,2-diamine with stirring; and
adding Co(NO₃)₂·6H₂O in situ followed by refluxing to yield the non-toxic mononuclear side-off compartmental cobalt(III) Schiff base complex C₂₄H₃₀N₃O₁₀Co.

Still another embodiment of the present invention is directed to provide said process for preparation of the Schiff base complex comprises the steps of:
providing ethanolic solution 5 ml to 10 ml preferably 5 ml of 2,6-diformyl-4-methylphenol 0.162 g (0.9878 mmol) to 0.166 g (1.0122 mmol) preferably 0.164 g, (1 mmol);
adding dropwise ethanolic solution of (±) cyclohexane-1,2-diamine 0.055 g (0.4824 mmol) to 0.0059 g (0.5175 mmol) preferably 0.057 g (0.5 mmol) with stirring;
adding Co(NO₃)₂·6H₂O (0.289 g (0.9931 m mol) to 0.293 g (1.0069 m mol) preferably 0.291 g, 1 m mol) in situ followed by refluxing for 22 h to 26 h preferably 24 h at 100° C. providing a dark brown-colored solution which was filtered and solvent evaporated off; and
washing the residual solid several times with ice cold water and then solubilized in methanol which upon keeping in desiccator provide dark brown crystals of the Schiff base complex.

### BRIEF DESCRIPTION OF THE DRAWINGS

The advancement according to the present invention is discussed in further detail in relation to the following non-limiting exemplary illustrations and accompanying figures wherein:

FIG. 1. Schematic representation of synthetic route to complex 1.

FIG. 2: Illustration of in vivo experimental processes for nontoxic dose determination.

FIG. 3. Molecular Structure of two independent units of complex 1 (nitrate anions, lattice water and hydrogen atoms not shown for clarity).

FIG. 4. Pair of units of Co2 in complex 1 connected by H-bonds (A similar pattern is observed for complex Co1).

FIG. 5: Cyclic voltammogram of complex 1 recorded in an aqueous medium.

FIG. 6. (a) The curve represents % cell viability versus different dose concentrations of the 1 and oxaliplatin (indicated in log value) in the MCF-7 cells after 24 h and 48 h treatment. The IC₅₀ value represents the mean±SD from the triplicate value of the absorbance from three independent experiments using graph pad prism (version 6). (b) The results of 1-way ANOVA and Tukey post hoc analysis to compare fold differences of mean normalized % viability values among various concentrations of complex 1 and oxaliplatin relative to untreated in MCF-7 cells. **P<0.01, ***P<0.001, ****P<0.0001 compared with the untreated control group treated with oxaliplatin and 1 with different concentrations of for both 24 h and 48 h. (c) The proliferation inhibition of MCF-7 cells after treatment with complex 1 was detected by an inverted phase-contrast microscope (magnification 10×; scale bar, 100 μM).

FIG. 6.1: Cytotoxicity analysis of complex 1 and oxaliplatin in different concentrations (5-40 μM) on HCT-116 and SW-480 for 24 h.

FIG. 7 (a) Annexin V-FITC/PI dual staining was used to determine the apoptotic rate in MCF-7 cells after 24 h of treatment with the IC₅₀ dose of oxaliplatin and complex 1. The Q4 quadrant (Annexin V−/PI−), Q3 quadrant (Annexin V+/PI−), Q2 quadrant (Annexin V+/PI+), and Q1 quadrant (Annexin V−/PI+) indicate the percentage of normal cells, early apoptosis, late apoptosis, and necrosis respectively. (b) Statistical analysis showed a significantly reduced percentage of the viable cells whether increased in early and late apoptotic population in both oxaliplatin and 1 treated cell with respect to untreated cells. *P<0.05**P<0.01, ***P<0.001, ****P<0.0001 compared between untreated control group with IC₅₀ dose of oxaliplatin and 1 treated group for 24 h. NS, P>0.05 represents no statistical significance between treatment groups of oxaliplatin and complex 1. All results are represented as mean±SD of three independent experiments. (c) Morphological alterations were seen in MCF-7 cells under higher magnification of 100× after DAPI staining (scale bar 100 mm). Yellow arrow pointed towards apoptotic cells.

FIG. 7.1: TUNEL assay showed the apoptotic cells (marked in yellow arrow) in HCT-116 cell line in Complex 1 and oxaliplatin-treated groups for 24 h. Cells were stained with DAPI and TUNEL and images were taken in fluorescence microscope (200× magnification).

FIG. 7.2: Induction of apoptosis in HCT-116 and SW-480. Cells were treated for 24 h with respective IC₅₀ dose of Complex 1 and oxaliplatin for both cell lines. Flow cytometric analysis by Annexin V/Propidium iodide dual staining showed that the percentage of early and late apoptotic cells were increased in complex 1-treated groups compared to untreated groups.

FIG. 7.3: The phase-contrast microscopic images showed that the treatment with respective IC₅₀ dose of complex 1 and oxaliplatin in HCT-116 and SW-480 reduced cell number; and apoptotic bodies were noticed in treated groups compared to untreated group (100× and 200× magnification).

FIG. 8 Induction of cell cycle arrest at the G2-M phase and inhibition of cell proliferation.

FIG. 8.1A and FIG. 8.1B: Anti-proliferative effect of Complex 1 and oxaliplatin on HCT-116 and SW-480 cells. Flow cytometric data showed that the treatment with IC₅₀ dose Complex 1 and oxaliplatin for 24 h decreased percentage of proliferative cells in Complex 1 and oxaliplatin-treated groups in comparison to untreated control group. The positive control group (cells cultured in serum-free media) showed least number of proliferative cells.

FIG. 8.2: Induction of cell cycle arrest at G0-G1 phase in HCT-116 and SW-480. Cells were treated for 24 h with respective IC₅₀ dose of Complex 1 and oxaliplatin for both cell lines. Flow cytometric analysis by propidium iodide staining showed that the percentage of cells in G0-G1 phase was considerably increased in Complex 1- and oxaliplatin-treated groups compared to untreated group for both cell lines. With significant rise in G0-G1 cell population there was concomitant decrease in cell population in S and G2-M phases.

FIG. 9 complex 1 induces apoptosis through G2-M arrest in MCF-7 cells. (a & c) Levels of G2-M phase arrest markers, p-Cdc^{Tyr15}, Cyclin B1 and p-Chk2^{Ser516}, Wee1, and p-CDC25c^{ser216} proteins were detected by western blot analysis where beta-actin was used as the loading control. (b & d) Statistical analysis of relative protein expression levels of G2-M phase arrest. Data represented as mean±SD of three independent observations where, **P<0.01, ***P<0.001, ****P<0.0001 compared with the untreated control group with treatment group of 1 (e) Representative photographs of western blot analysis of cell proliferation marker PCNA and apoptotic related proteins P53, Bax, Bcl2 and Bcl-xL where beta-actin was used as the loading control. (f) The relative protein expression levels of apoptotic markers were analyzed. Data represented as mean±SD of three independent observations where ***P<0.001, ****P<0.0001 compared with untreated control group with treatment group of 1.

FIG. 9.1 Western blot analysis of cell cycle related markers in HCT-116 and SW-480 after 24 h treatment with IC₅₀ of complex 1. The complex 1 induced cell cycle arrest at G0-G1 phase. The key regulators of G0-G1 phase, Cyclin D1 and its associated two CDKs, CDK4 and CDK6 were found to be downregulated in treated group in comparison to untreated control group.

FIG. 9.2: Western blot analysis of apoptosis associated markers in HCT-116 and SW-480 after 24 h treatment with IC₅₀ of complex 1. The expression of anti-apoptotic protein Bcl-xL was decreased and that of pro-apoptotic proteins Bax and Bim were increased in treated group compared to untreated group. Further, the expression of cleaved Caspase 3 and cleaved PARP were increased in treated group compared to untreated group.

FIG. 10: Effect of complex 1 and oxaliplatin on MCF-7 cell migration in vitro. (a) Schematic drawing of the process of making scratches. The cultured confluent MCF-7 cells monolayer (right drawing) was scratched using a pipette tip. (b) Images of the wound closure were captured using a 10× (scale bar, 100 μM) magnification. The limits of the wound are indicated by yellow dotted lines at the beginning of the assay and 12 h, 24 h, and 48 h after scratching. (c) Mean wound area percentages for the oxaliplatin and 1 treated groups at various time points were compared to the corresponding untreated control group. According to statistical analysis, untreated cells had a considerably higher proportion of the healed area compared to oxaliplatin and complex 1 treated cells.****P<0.0001 compared between untreated control group with IC₅₀ dose of oxaliplatin and 1 treated group. NS represents no statistical significance between treatment groups of oxaliplatin and 1. All results are represented as mean±SD of three independent experiments.

FIG. 10.1: Anti-migratory effect of Complex 1 and oxaliplatin on HCT-116 and SW-480. Cells were treated with respective IC₅₀ dose of Complex 1 and oxaliplatin for both cell lines. The time point of creating the scratch was considered as 0 h. After treatment, the images of corresponding scratch area were captured at 12 h, 24 h, and 48 h. It was observed that the scratch area (gap) became small in untreated group than the treatment groups for both cell lines. This showed that Complex 1 reduced the migration of HCT-116 and SW-480 cells in a time-dependent manner and was comparable with that of the oxaliplatin-treated group. The scratch area was represented with the yellow line.

FIG. 11. Representative histological sections of liver tissue from Swiss albino mice treated with complex 1 obtained on 28^{th} day for the subacute toxicity and 40^{th} day for chronic toxicity study (a) H&E staining of liver sections under the lower magnification of 10× is shown in the upper row (scale bar 200 μm) and a higher magnification of 40× is shown in the lower row (scale bar 20 μm) to highlight the findings. (A, B) Untreated liver tissue consists of radially arranged hepatocytes (HC) with well-demarcated regular size hepatocyte nuclei (HN), hepatocyte intervening sinusoids (S), normal central vein (CV). (C, D) Liver tissue after the 28^{th} days treatment showing normal hepatocyte (HC), portal vein (PV), intervening sinusoid (S). The radial distribution pattern around the central vein was prominent and no neutrophil infiltration. (E, F) Radially arranged hepatocytes (HC) with a smaller number of Kupffer cells (KC) in the liver tissue after 40^{th} days of treatment. (b) Reticulin staining of liver section sample from the untreated group (A, C, E) and 40^{th} days treated group (B, D, F) was observed under magnification 4×, 10× and 40×. (B, D,) The lining around the sinusoidal portal tract (PT) and hepatic venules (V) were reticulin positive (reticulin fibers are black, shown in yellow arrow). (F) After the 40^{th} day of treatment, the intracellular spaces (indicated in yellow arrow) of the liver tissue are reticulin negative.

FIG. 12. Representative histological sections of kidney tissue from Swiss albino mice treated with complex 1 obtained on 28^{th} day for the subacute toxicity and day 40^{th} for chronic toxicity study (a) H&E staining of kidney tissue sections under the lower magnification of 10× is shown in the upper row (scale bar 200 μm) and a higher magnification of 40× is shown in the lower row (scale bar 20 μm) to highlight the findings. (A, B) Untreated kidney tissue consists of the relatively healthy glomerulus (G) with abundant capsular space and normal renal corpuscles (RC) and glomerular tufts. (C, D) 28^{th} days treated Kidney tissues showing no glomerular infiltration, tubular (proximal convoluted tubule, PCT) dilation, necrosis, and enlargement of epithelial cells lining of the renal tubules. (E, F) Normal renal corpuscles (RC) and glomerular tufts (G) of kidney tissue after 40 days of treatment. (b) PAS staining of kidney tissue sections was observed under 40× magnification (scale bar 20 μm) on the 40^{th} day. (B) There was no glomerular basement membrane (GBM) thickening and absence of material deposition in mesangial kidney tissue of the treatment group.

FIG. 13: Schematic diagram of the present invention (for MCF-7).

### DETAILED DESCRIPTION

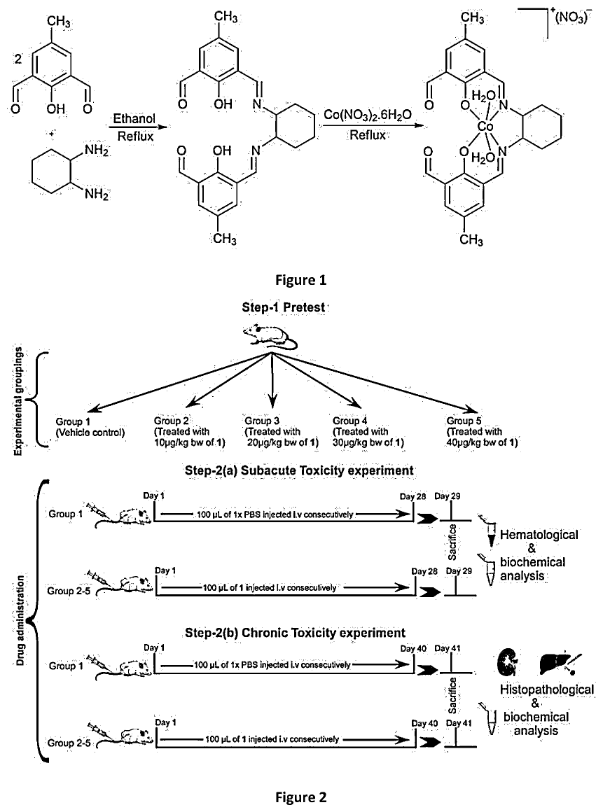

The present invention relates to in situ synthesis of a novel mononuclear side-off compartmental cobalt (III) Schiff base complex (1) using cyclohexane-1,2-diamine, 2,6-diformyl-4-methylphenol and Co(NO₃)₂·6H₂O (FIG. 1) and its structural analysis along with in-vitro cytotoxicity study against human breast cancer cell line (MCF-7), Human colon cancer cell lines HCT-116, SW-480 and human PBMCs in comparison to the activity of oxaliplatin as standard reference drug. The results from the in vitro study showed that the complex effectively induced apoptosis in MCF-7 cells via G2-M phase cell cycle arrest and in a different pathway in colon cancer cell lines HCT-116 and SW-480 via G0-G1 cell cycle arrest. The toxicity study of complex 1 is also carried out in normal Swiss albino mice to see whether there is any adverse side effect on the host systems. Hematological, biochemical, and histopathological analyses of the organs were conducted to measure the systemic toxicity parameter, which may prove to be useful for its future applications as chemotherapeutics.

As stated hereinbefore that a few Cobalt Schiff base complexes exhibiting cytotoxicity against cancer cells may act as a good alternative to cisplatin but detailed efficacy study and toxicity profiling are needed to project them as future chemotherapeutics.

The aldehyde 2,6-diformyl-4-methylphenol has intense fluorescence property. So, employing this aldehyde in the production of the compound would have the advantage for tracking the drug. As anticipated the invented complex also shows good fluorescence sensitivity. Apart from this property, as stated in the background that several complexes with different metal ions like Co(II), Ni(II), Cu(II), Zn(II), Hg(II), Mo(VI) and Pd(II) containing the Schiff base ligand derived from 2,6-diformyl-4-methylphenol showed antiproliferative behavior against the five human tumor cell lines like human neuroblastoma (NB69), human breast cancer (MCF-7, EVSA-T), human glioma (H4), and human bladder carcinoma cell line (ECV) (2). Keeping this backdrop in mind, 2,6 diformyl-4-methyl phenol was selected for imine preparation. However the synthetic procedure adopted and product of the present application is different as compared to the prior art.

Synthetically the reported complex is with end-off type ligand and the ultimate complex is made in stepwise manner whereas the invented one in accordance to the present invention is prepared via one pot synthetic method resulting in side-off ligand metal complex. Present Schiff base is made with the molar ratio of 1:2 (amine:aldehyde) in contrast to the ratio of 2:1 (amine:aldehyde) for the reported complex, however no NMR/crystal structure data of the cobalt complex is reported whereas present complex is well characterized via NMR and single crystal XRD study. In the said publication only colorimetric cytotoxicity assay (CCA) data is provided against the human neuroblastoma NB69 cell line, and IC₅₀ for breast cancer cell lines is only mentioned to be similar. No other studies were done to justify anticancerous properties of the cobalt Schiff base complex or toxicological property towards the normal host systems or to establish the molecular mechanism behind the activity. Therefore, the work done in the said reference failed to teach the side off compartmental cobalt Schiff base complex as nontoxic chemotherapeutic agent in breast cancer and colon cancer cell lines.

Choice of 1,2-diaminocyclohexane (DACH) was motivated by oxaliplatin, a third-generation platinum based anticancer drug. It is well known that due to presence of DACH, platinum DNA adduct formed with oxaliplatin are bulkier and more hydrophobic and thereby making DNA repair mechanism more difficult and thus showing higher efficacy of oxaliplatin than other platinum medications. As DACH reported to interact with cation transporters present in the cell membrane this feature may facilitate the compound's cellular entry. For the present invention DACH is condensed with an aldehyde 2,6-diformyl-4-methylphenol for the production of the Schiff base complex to make the ligand system even bulkier. The bulkier ligand may also have a positive role to play by not crossing the blood brain barrier because, after chelation with the metal it increases the molecular weight of the compound beyond 400 Da, therefore may have none or very less central nervous system (CNS) toxicity.

Idea of selecting cobalt as a replacement of Platinum is inspired by Doxovir, a cobalt (III) Schiff base complex (1:2 amine:aldehyde ratio), which has progressed to phase-II antiviral clinical trials (Schwartz, J A et al., Herpes simplex virus type 1 entry is inhibited by the cobalt chelate complex CTC-96. J Virol. 2001 May; 75(9):4117-28). The mechanism of action behind its antiviral property is the dissociative exchange of its labile 2-methylimidazole with histidine residues of herpes virus serine protease (essential for viral replication). The derivative of doxovir has been further used to inhibit histidine residues in ZFTFs (Zinc finger transcription factors) involved in cancer progression (viz, Snail and Hedgehog signaling)(doi: https://doi.org/10.1128/JVI.75.9.4117-4128.2001; https://doi.org/10.1021/mp2005577; https://doi.org/10.1371/journal.pone.0032318). Therefore, it was presumed that it would be advantageous to synthesize novel cobalt(III) Schiff base that could be used as potential chemotherapeutics to target pathways related to cancer if the same can aim precisely the transcription factors involved in tumor progression and metastasis.

Thus the primary embodiment of the present invention provides a novel Cobalt Schiff base complex synthesized by using the 1,2-diaminocyclohexane and 2,6-diformyl-4-methylphenol, Co(NO₃)₂·6H₂O characterized it and isolated as crystalline solid with >99% purity. This newly synthesized complex 1 under investigation, at first place, checked for its cytotoxicity against the human breast cancer cell line MCF-7 (MTT assay) and Human colon cancer cell lines HCT-116 and SW-480 (XTT assay), keeping the common metallodrug oxaliplatin as reference standard at different time points (24 h and 48 h), was found to restrict cell proliferation which was further validated by CFSE staining assay. Again, it induced apoptosis to the same degree as oxaliplatin at both the early and late stages, as confirmed by AnnexinV-FITC/PI-PE staining assay for the human breast cancer cell line MCF-7. Cell cycle distribution analysis revealed that both complex 1 and oxaliplatin arrest the cell cycle to a similar extent at the G2-M phase. Additionally, complex 1 inhibits cell migration to almost similar extent as in the case of oxaliplatin.

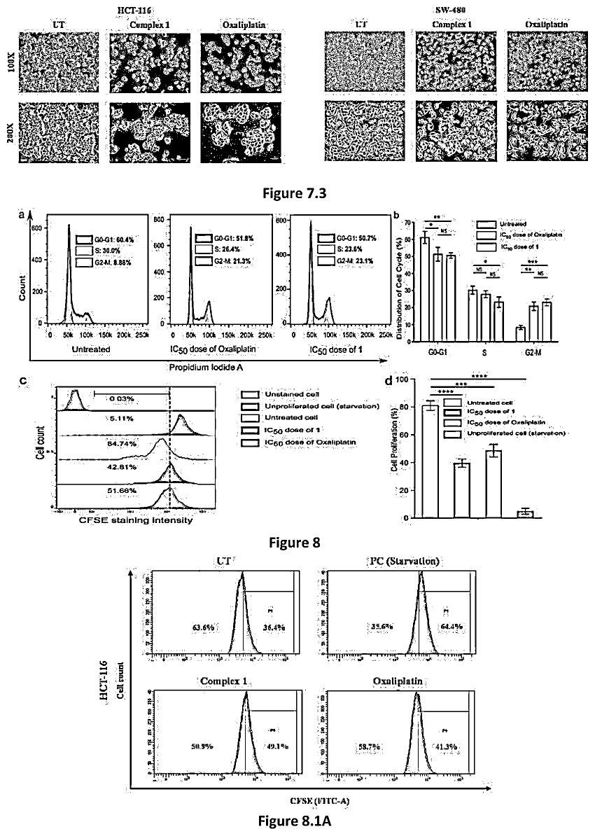

Similarly, XTT assay revealed that complex 1 exhibits cytotoxic effects on HCT-116 and SW-480 cells after 24 h of incubation with IC₅₀ values 15.27±1.18 μM and 10.04±1.98 μM respectively. The IC₅₀ values of the complex 1 were comparable with the IC₅₀ values of oxaliplatin which was 16.73±1.78 μM and 7.87±1.54 μM respectively for HCT-116 and SW-480 (FIG. 6.1)

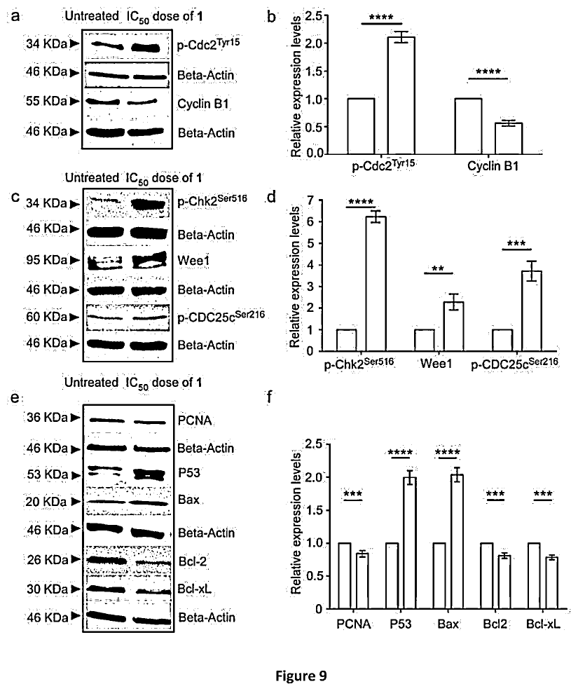

Flow cytometric analysis of cell proliferation (using CFSE) and cell cycle distribution (using PI) showed that 24 h treatment with respective IC₅₀ dose of complex 1 and oxaliplatin suppressed the cell proliferation in both the cell lines. The antiproliferative efficacy of complex 1 was more than that of oxaliplatin in HCT-116 but was comparable in SW-480 (FIG. 8.1). The population of cells in G0-G1 phase was increased with concomitant decrease in S and G2-M phase, indicating the cell cycle arrest at G0-G1 phase in both HCT-116 and SW-480 after 24 h treatment with respective IC50 dose of complex 1 and oxaliplatin. In both HCT-116 and SW-480, the G0-G1 arrest was non-significant in complex 1-treated group and oxaliplatin-treated group. Therefore, complex 1 and oxaliplatin arrested cell cycle in a similar extent (FIG. 8.2).

In another embodiment side-by-side toxicity assessment of 1 was performed on normal human PBMCs to establish the dosage limit for a new drug compound where the compound did not cause any significant cytotoxicity even at higher concentrations. While examining a new drug candidate intended for human use, safety is the top most priority. Therefore, before exploring the underlying molecular mechanism behind the compound's activity in vivo systemic subacute (28 days) and chronic (40 days) toxicity studies were conducted on mice. The results suggest that complex 1 with 10 μg/kg-40 μg/kg of body weight of doses does not impart any significant hematological toxicity, nephrotoxicity, or hepatotoxicity (biochemical analysis). The highest non-toxic dose of 1 was applied for histopathological analysis to the kidney and liver tissues demonstrating that treatment with this dose has no observable damage to the mice's vital organs.

In another embodiment the present invention further investigated the underlying molecular mechanisms responsible for the compound's activity. Apoptosis plays a key role in the balance between survival and death in multicellular organisms and in maintaining homeostasis in mammalian cells. Evading apoptosis increases abnormal cell growth or proliferation, which ultimately results in cancer. A number of studies suggest that p53 protein causes cell death by suppressing the expression of the anti-apoptotic protein Bcl2, Bcl-xL, which causes the pro-apoptotic protein Bax to disassociate from heterodimeric complexes of Bax/Bcl-2 or Bax/Bcl-xL. After that, oligomeric Bax proteins form lipid holes in the mitochondrial outer membrane, through which apoptosis-inducing stimuli have been released [Dashzeveg, N., et al., Cell death decision by p53 via control of the mitochondrial membrane, Cancer Lett. 367 (2015) 108-112]. The expression of cell proliferating marker PCNA has been implicated as a potential biomarker for breast cancer. The western blot analysis revealed that complex 1 elevated tumor suppressor protein P53 levels and apoptotic protein Bax while decreasing anti-apoptotic protein Bcl2 and Bcl-xL expression level and cell proliferating marker PCNA, confirming that cell death is mediated by apoptosis.

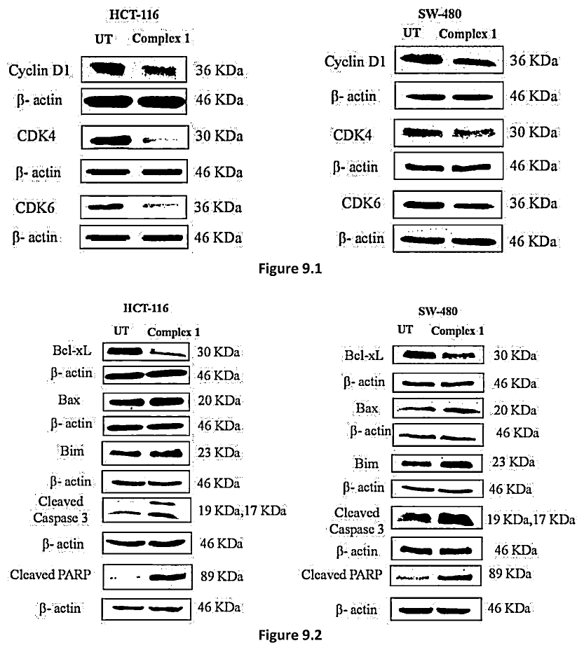

In another embodiment the protein expression of cell cycle inhibitors and other cell cycle-associated proteins was checked for understanding the signaling mechanism of complex 1 responsible for the cell cycle inhibitory effect. The G2-M phase regulatory proteins Cyclin B1 and p-Cdc2Tyr15 were examined using western blotting to confirm the results of cell cycle analysis. The master regulator for the M-phase transition is Cyclin B1, which is one of the main protein kinases that become activated. On the other hand, activation of the cdc2 kinase is required for the regulatory subunit Cyclin B1 to bind to Cdc2 (also known as CDK1) and form MPF. However, phosphorylation at Tyr15 inhibits active Cdc2 activity, preventing cells from entering the M phase. In the present invention elevated level of p-Cdc2Tyr15 expression, while downregulated Cyclin B1 expression were observed. Interestingly, the Cdc2/Cyclin B1 complex remains inactive by phosphorylation of Cdc2 protein on tyrosine 15 by the kinases Wee1 at the G2 phase. At the beginning of the transition from the G2 to the M phase, Cdc25C phosphatases remove the phosphate residue from tyrosine 15. Therefore, the elevated Cdc25C phosphatases and downregulated wee1 proteins are essential for the G2-M phase transition. The Chk2-dependent pathway is thought to be crucial in the response to DNA damage, where phosphorylation of Chk2at Ser 516 leads to phosphorylation of Cdc25C at Ser 216, thus, inactivating the phosphatase activity of Cdc25C. These results suggested that complex 1 successfully promoted G2-M phase cell cycle arrest for MCF7 cells by preventing the formation of the Cdc2/Cyclin B1 complex, by elevating the expression level of Wee1, p-Chk2^{Ser516} and p-CDC25C proteins. (FIG. 9). Human colon cancer cell lines HCT-116 and SW-480 after 24 h treatment with complex 1 and oxaliplatin showed increase in the population of cells in G0-G1 phase with concomitant decrease in S and G2-M phase, indicating the cell cycle arrest at G0-G1 phase (FIGS. 9.1 and 9.2)

It is to be understood that both the foregoing general description and the following detailed description are exemplary, and are intended to provide further explanation of the invention directed to the advance of a significantly non-toxic novel Cobalt(III) Schiff base complex capable to induces apoptosis via G2-M cell cycle arrest in human breast cancer cell line MCF-7.

### Abbreviations Used

ESI-MS, Electrospray ionization mass spectrometry; FT-IR, Fourier transform infrared; UV-Vis, Ultraviolet-visible spectroscopy; HPLC, High performance liquid chromatography; MTT, 3-(4,5-dimethylthiazol-2-yl)-2,5-diphenyl tetrazolium bromide; PBMCs, Peripheral blood mononuclear cells; IC₅₀, Half maximum inhibitory concentration; CFSE, 5-(and 6)-Carboxyfluorescein diacetate succinimidyl ester; PCNA, Proliferating cell nuclear antigen; p-Cdc2^{Tyr15}, Phosphorylated cell division control protein 2; MPF, Maturation promoting factor; P-CDC25C^{Ser216}, Phosphorylated cell division cycle 25C; p-Chk2^{Ser516}, Phosphorylated check point kinase 2; CDK1, Cyclin dependent kinase 1; Bax, Bcl-2-associated X protein; Bcl2, B-cell lymphoma 2; Bcl-xL, B-cell lymphoma-extra large; RBCs, Red blood cells; WBCs, White blood cells; PLTs, Platelets; HGB, Haemoglobin; ALP, Alkanine phosphatase; SGOT, Serum glutamic-oxaloacetic transaminase; SGPT, Serum glutamic pyruvic transaminase; H&E staining, Haematoxylin and Eosine; PAS, Periodic acid Schiff; FBS, Fetal bovine serum; PBS, Phosphate buffer saline; DMSO, Dimethyl Sulfoxide; BSA, Bovine serum albumin; BCA, Bicinchoninic acid; SDS-PAGE, Sodium dodecyl sulphate polyacrylamide gel electrophores; APS, Ammonium per sulphate; PI cocktail, Protease inhibitor cocktail; TEMED, N,N,N′,N′-Tetramethylethylenediamine; PVDF, Polyvinylidene difluoride; TBST, Tris-buffered saline tween-20; ECL, Enhanced Chemiluminescence; RIPA buffer, Radioimmunoprecipitation assay buffer; HRP, Horseradish peroxidase.

### EXAMPLES

### Reagents/Chemicals and Antibodies

All the reagents and chemicals were purchased from Merck, S. D. Fine-Chem Limited, or Sigma Limited unless otherwise noted, and used without further purification. Solvents were dried according to standard procedure and distilled before use. Water used in all physical measurements and experiments was Milli-Q grade. For biological experiments, RPMI-1640 Medium, FBS, trypsin-EDTA, non-essential amino acids, and antibiotics (penicillin-streptomycin, antibiotics antimycotics) were procured from Gibco (USA). MTT, 1×PBS, cell culture grade DMSO, molecular biology grade water, BSA, hematoxylin and cosin were obtained from Himedia (Mumbai, India). XTT reagent kit was purchased from R & D systems. Propidium iodide, Histopaque 1077, RIPA buffer, PI cocktail, Trizma, SDS, APS, Tween 20, Bromophenol blue, Glycine, RNAase, Reticulum staining kit, and PAS kit were bought from Sigma. TEMED, Glycerol, Xylenes, and 37% formaldehyde solution were purchased from Merk. BCA protein assay kit and FITC Annexin V/dead cell Apoptosis kit were procured from Thermofisher Scientific. Trans-Blot Turbo RTA mini 0.2 μm nitrocellulose transfer kit, 30% acrylamide and bis-acrylamide solution, Precision Plus Protein dual-color standards, Clarity Max ECL Western Blotting Substrates were purchased from Biorad Laboratories. PVDF membrane was purchased from Millipore. Bax, Bcl-XL, P53, Cyclin B1, p-Chk2^{Ser516}, p-Cdc2^{Tyr15}, p-CDC25c^{Ser216}, Wee1, Bim, Cyclin D1, CDK4, CDK6, cleaved caspase 3, cleaved PARP, Anti-rabbit, and anti-mouse IgG, HRP-linked antibodies were purchased from Cell Signalling Technology. Bcl2, PCNA, and beta-actin antibodies were procured from BD biosciences, Santa Cruz biotechnology, and Thermofisher Scientific respectively. CFSE cell division tracker kit was purchased from Biolegend. All the other chemicals used for the experimental purpose are of analytical reagent grade and were obtained from common commercial sources.

Statistical analysis: All the experiments of in-vivo and in-vitro models were performed in triplicate for the reproducibility of the data and the data reported in this communication were expressed as the arithmetic mean±SD (standard deviation) and the graphs were plotted using Prism 6 (Version 6.0b, GraphPad Software). For in-vivo study, the number of mice per group is six (n=6). The inhibitory concentration of 1 in which 50% cell growth was inhibited (IC₅₀) was determined using Prism Graph pad 6 software. One-way ANOVA and Tukey post hoc test has done to evaluate the mean % viability value between untreated and different concentrations of treated groups in MCF-7 cells. The unpaired Student's t-test was performed to analyze all the in-vivo toxicological data and the P-value<0.001 was considered statistically significant (***) in the entire results.

### Example-1

### Synthesis of the Schiff Base Ligand and Complex 1

To an ethanolic solution (5 mL) of 2,6-diformyl-4-methylphenol (0.164 g, 1 mmol), ethanolic solution of (±) cyclohexane-1,2-diamine (0.057 g, 0.5 mmol) was added dropwise with continuous stirring. Stirring was continued for 3 h. To the ethanolic solution of ligand Co(NO₃)₂·6H₂O (0.291 g, 1 mmol) was added in situ and the reaction mixture was refluxed for 24 h at 100° C. A dark brown-colored solution was formed and was filtered. The crude solid product obtained after evaporation was washed several times with ice cold water and then solubilized in methanol. The methanolic solution of the complex was kept in desiccators and dark brown crystals of complex 1 suitable for single crystal x-ray diffraction were obtained after a week. Elemental analyses (carbon, hydrogen, and nitrogen) were performed using a Perkin Elmer 240 C analyzer. Elemental Anal. Calc. (%) for C₄₈H₅₆N₆O₁₈Co_{2 (}In the crystal structure, two asyemmtric units of the complex are present in close proximity in a single unit cell and are connected by two hydrogen bonds—which is reflected in elemental analysis providing MF of dimeric form): C 51.34; H 5.03; N 7.48; Found (%): C 51.26; H 5.07; N 7.46; ESI-MS (H₂O): m/z=463.0116 amu; HPLC (DMSO-H₂-O): displayed>99% purity; FT-IR (KBr): ν(C═N) and ν(C═O) 1642 cm⁻¹(broad); ν(skeletal vibration) 1550 cm⁻¹; ν(NO₃⁻) 1385 cm⁻¹; UV-Vis (MeOH): λₘₐₓ=410 nm, 610 nm; UV-Vis (0.03% DMSO-H₂O): λₘₐₓ(ε)=412 nm, 354 nm.

### FT-IR Spectral Study of Complex 1

Infrared spectrum (4000-400 cm−1) of coplex 1 was recorded at 28° C. on a 6 Perkin-Elmer RXI FT-IR spectrophotometer using KBr as a medium and contains a broad band consisting of two small breaks at 1634 cm⁻¹ and 1642 cm⁻¹ due to the presence of C═N and free C═O. The sharp bands at ˜1550 cm⁻¹ and ˜1385 cm⁻¹ are present due to skeletal vibration and nitrate ion.

### UV-Vis Spectral Study of Complex 1

UV-Vis spectrum of complex 1 was recorded in Shimadzu UV-3101PC instrument and within the range of 800-300 nm with methanol and 0.03% DMSO-H₂O solvent and reference. The spectral study of complex 1 in MeOH medium shows a strong band at λₘₐₓ˜410 nm, whereas in 0.03% DMSO-H₂O solution two bands were observed at λₘₐₓ˜412 nm and 354 nm due to ligand to metal charge transfer. To monitor the stability of complex 1 in 0.03% DMSO-H₂O medium UV-Vis spectrum were recorded at a regular interval of 24 h, for 7 days with the same solution and it is found that the complex 1 is stable up to one week.

### X-Ray Single Crystal Study

Diffraction intensity data for crystal structure analysis of complex 1 was collected at room temperature with Mo-Kα radiation (λ=0.71073 Å) on a Bruker Smart Apex diffractometer equipped with CCD. Cell refinement, indexing, and scaling of the data sets were performed using the program Bruker Smart Apex, and Bruker Saint packages. The structure was solved by direct methods and subsequent Fourier analyses and refined by the full-matrix least-squares method based on F² with all observed reflections. Hydrogen atoms were placed at calculated positions (except those of aquo ligands located on the Fourier map) and included in the final cycles of refinement. Both nitrate anions share their position with a lattice oxygen water (at half occupancy), but no H atoms were assigned to the latter molecules, but included in the formula. All calculations were performed using the WinGX System, Ver. 2018.3. Crystal data and details of the refinement are presented in Table 1.

TABLE 1
Crystal data and details of the structure
determination for complex 1

| | |
|---|---|
| CCDC Number | 1992856 |
| Empirical Formula | C₂₄H₃₀N₃O₁₀Co |
| Formula Weight | 579.44 |
| Crystal System | Orthorhombic |
| Space group | C 222 |
| a (Å) | 11.6769 (10) |
| b (Å) | 19.8456 (10) |
| c (Å) | 22.8210 (13) |
| V (Å³) | 5288.4 (6) |
| Z | 8 |
| Dcalc (g/cm³) | 1.450 |
| μ (Mo Kα) (mm) | 0.709 |
| F(000) | 2400 |
| Crystal size (mm) | 0.29 × 0.32 × 0.30 |
| θ min, θ max (°) | 0.9, 26.4 |
| Tot., Uniq. reflections, Rᵢₙₜ | 35814, 5440, 0.0424 |
| Observed data (I > 2σ(I) | 4385 |
| Parameters refined | 391 |
| R1, wR2, S | 0.0355, 0.0922, 1.059 |
| Absolute structure parameter | 0.003(16) |
| Residuals e.Å⁻³ | 0.333, −0.241 |

w = 1/[S²(Fo²) + (0.0658P)² + 1.5358P], Where P = (Fo² + 2Fc²)/3.

Crystallography Study of [Co(L)·(H₂O)₂]NO₃·H₂O (Complex 1)

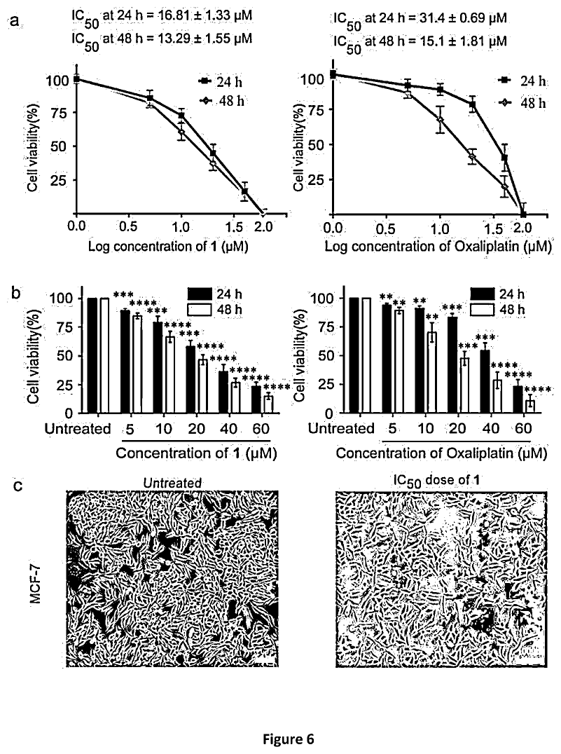

The X-Ray crystal structure analysis of complex 1 revealed that it crystallizes in orthorhombic C222 space group, and the asymmetric unit comprises two comparable complex units with half ligand being the metal (Co1, Co2) located on a crystallographic 2-fold axis counterbalanced by two nitrate anions at half occupancy. In each species the cobalt is chelated in the equatorial plane by two symmetry related phenoxide oxygen atoms and imine nitrogen donors of the tetradentate dianionic ligand. The metals complete the coordination sphere with two water molecules at the axial positions. Thus the crystallographic analysis reveals that the two cobalt(III) ions are hexacoordinated in a distorted octahedral geometry (FIG. 3) as evident from bond angles. The two complex units show comparable coordination parameters within their esd's. In fact, the Co—O(phenoxo) bond distances are of 1.887(3) Å and 1.880 (3) Å and Co—N(imine) are of 1.888(4) Å and 1.901(4) Å for unit 1 and unit 2, respectively. The two symmetry related water molecules at axial positions show Co—O(water) bond lengths of 1.912(3) Å and 1.917(3) Å for complex 1 and unit 2, respectively, which are slightly longer than the Co—O(phenoxo) bond values, indicating a slight tetrahedral distortion in the octahedral geometry.

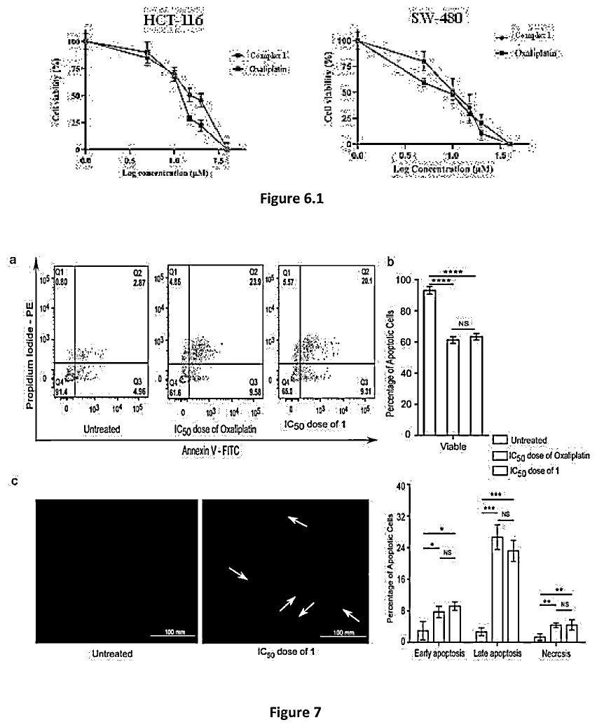

The two complex units are paired and mutually hydrogen-bonded as shown in FIG. 4. Thus, in the crystal packing the complexes are connected by hydrogen bonding occurring between coordinated water molecules and aldehyde O atoms of the other unit, O_{1w}-H_{1w} . . . O₂ (with O . . . O distance=2.596(6) Å) and O₂w-H_{4w} . . . O₄ (O . . . O distance=2.615(6) Å), which strongly stabilizes the assembly.

### High Performance Liquid Chromatography (HPLC) Study

HPLC was performed in Agilent Prep 1290 Infinity II HPLC System using Agilent Prep 1290 Infinity II pump and DAD 1260 Infinity II detector and run on an C-18, Reserve Phase Column from WATERS (4.6×150 mm, particle size 5um), using 0.1% TFA in H₂O and 100% CH₃CN as mobile phase. At retention time 2.018 min, a single peak of complex 1 in DMSO-H₂O medium was detected with area percentage of 99.17 through HPLC analysis. As the complex displayed >99% purity, we further proceed with the biological evaluation of that complex.

### ESI-MS Study

The mass spectrum of complex 1 was recorded in a Waters Xevo-G2 S Q-Tof mass spectrometer in an DMSO-H₂O medium and positive mode to have an insight about the solution phase structure. The base peak with 100% abundance is located at m/z=463.0116 amu which corroborates well with molecular formula C₂₄H₂₄CoN₂O₄⁺ (Calcd. m/z=463.0168 amu). Another peak is observed at m/z=499.1307 amu and this peak matches well with the complex cation having molecular formula C₂₄H₂₈CoN₂O₆⁺ (Calcd. m/z=499.1279 amu). Hence it is confirmed that 1 retains its mononuclear entity in the solution phase.

### Cyclic Voltammetry Study

The cyclic voltammetry experiment of complex 1 was performed in a Basi C-3 cell voltameter under nitrogen with a glassy carbon electrode as the working electrode, Ag/AgCl as the reference electrode, and tetrabutylammonium perchlorate as the supporting electrolyte.

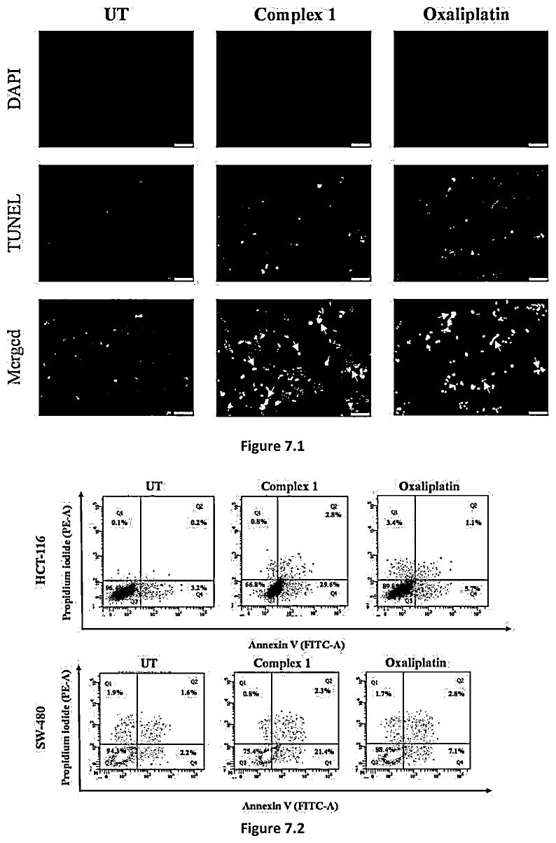

The cyclic voltammetry (CV) study was performed to ensure the oxidation state of cobalt in complex 1 and was carried out in an aqueous medium at a scan rate of 100 mV/sec. The Co^{III}/Co^{II} redox couple appears at −0.93 V with respect to Ag/AgCl reference electrode and the cathodic peak at −1.13 V is attributed to Co^{II}/Co^{I} redox couple as shown in FIG. 5. The other cathodic peak at −1.7 V is attributed to the reduction of C═N linkage of the Schiff base ligand. Hence it is proved that cobalt is present in the +3 oxidation state in complex 1.

### Example-2

### Samples With Different Concentration (Dosage Form) Preparation for Both In-Vitro and In-Vivo Experiments

For the in-vitro cytotoxicity study, 1.2 mg of complex 1 (MW: 579.12) was dissolved in 30 μL cell culture grade DMSO and then diluted to 2 mL by adding RPMI-1640 medium containing 10% FBS to prepare a 0.6 mg/mL stock solution (0.001M). Then this stock solution was serially diluted with FBS containing RPMI-1640 medium to obtain the concentration (5 μM, 10 μM, 20 μM, 40 μM, and 60 μM) required for subsequent experiments. Similar to this, 1.3 mg of oxaliplatin was dissolved in 92.85 μL of DMSO before being gently mixed with 907.15 μL of RPMI-1640 medium to produce a stock concentration of 3.2 mM. Drugs of various concentrations (5 μM, 10 μM, 20 μM, 40 μM, and 60 μM) were made from this stock solution through serial dilution. For in-vivo studies, 1 mg/mL stock solution (made in the same way as mentioned above, where molecular biology grade water is used instead of RPMI-1640 medium) of this complex was serially diluted with phosphate buffer saline (PBS) to get the desired concentration (10 μg/kg, 20 μg/kg, 30 μg/kg, and 40 μg/kg body weight of mice). For in-vitro studies, 0.09% DMSO with RPMI-1640 medium was used as vehicle control. On the other hand, for in-vivo experiments, 1×PBS was injected intravenously (i.v) as vehicle control.

### Cell Lines and Culture Conditions

Breast cancer cell line and culture condition: Cells were cultured in a humidified air-jacked CO₂ incubator (Thermo scientific, Heracellvios 160i). Live cancer cells were viewed and cell migration assay was carried out by a cell culture inverted microscope (Olympus, CKX 41). Human breast cancer cells lines MCF-7, were purchased from NCCS, Pune. Invasive ductal carcinoma cells (MCF-7) lack caspase-3, and exhibit wild-type p53 and estrogen receptors. The cells were cultured as monolayers in RPMI-1640 supplemented with 10% heat-inactivated fetal bovine serum (FBS), 1% penicillin-streptomycin, and 1% antibiotic-antimycotics and 1% non-essential amino acids at 37° C. in a fully humidified air-jacketed incubator of 5% CO₂ and 95% air. All the experiments were performed from exponentially growing passage of 15 or less with 80% cell confluency and then sub-cultured to a fresh media to keep cells healthy and actively growing.

Colon cancer cell lines and culture condition: Human colon cancer cell lines HCT-116 and SW-480 were purchased from NCCS, Pune. The carcinoma cell line (HCT-116) is wild-type for TP53 and adenocarcinoma cell line (SW480) is a double-mutant for TP53 gene. The cells were cultured as monolayers in RPMI-1640 medium supplemented with 10% heat-inactivated fetal bovine serum (FBS), 1% of each penicillin-streptomycin, antibiotic-antimycotic, sodium pyruvate and non-essential amino-acid at 37° C. in a fully humidified air-jacketed incubator of 5% CO₂. All the experiments were performed from exponentially growing passage of 15 or less with 80% cell confluency and then sub-cultured to a fresh media to keep cells healthy and actively growing.

Cytotoxicity study: MTT assay was done in a laminar flow clean air work station (Klenzaids, 1194). Cell survival was analyzed by an ELISA plate reader (Tecan M200). Live cancer cells were viewed and cell migration assay was carried out by a cell culture inverted microscope (Olympus, CKX 41). The BD FACS Verse, Becton Dickinson, San Jose was used to study cell cycle and apoptosis. Fluorescence microscope (Olympus, BX 63) was used to notice the morphological changes of the cancer cells after drug treatment.

MTT assay: To determine the cytotoxicity of complex 1 and standard reference drug oxaliplatin on breast cancer cells, the MTT assay was conducted separately. The MTT assay is a quantitative, sensitive, and reliable colorimetric assay that mainly measures cell viability and proliferation. Cytotoxicity studies of both complex 1 and oxaliplatin were performed by seeding into 96-well microtiter plates at a density of 1×10⁴ cells/well in 100 μL of culture medium and then incubated for 24 h for adherence. Subsequent overnight incubation, culture media was changed by 200 μL of media with complex 1 and oxaliplatin at a varying concentration of 5 μM, 10 μM, 20 μM, 40 μM, and 60 μM respectively for 24 and 48 h. Each plate contained a blank, vehicle control, treated and untreated samples, and media containing 0.09% DMSO was used as vehicle control. The culture medium was changed by adding 90 μL fresh culture medium and 10 μL of sterile-filtered solution (5 mg/mL) in phosphate-buffered saline (PBS, pH=7.4) to each well and incubated at 37° C. for 4 h. After 4 h, the remaining unconsumed dye was removed, and to ensure total solubility of the insoluble formazan crystals, 100 μl DMSO was added to each well. The cells viability was estimated spectrophotometrically by measuring absorbance at 570 nm using an ELISA plate reader. To calculate the cell viability percentage, the average absorbance of each treatment group was acquired and normalized based on the untreated control. The half inhibitory concentration (IC₅₀ value) was calculated by putting the experimental data with the dose-response model (Prism 6, Version 6.0b, GraphPad Software). Besides, human peripheral blood was heparinized and diluted with RPMI-1640. After centrifugation, the lymphocyte-enriched mononuclear cells were resuspended in cold RPMI-1640 media with 10% heat-inactivated FBS, 2 mM L-glutamine, 1% penicillin/streptomycin, and 1% antibiotic/antimycotics. The same MTT experiment was done on normal human PBMCs cultured for 24 h. It was calculated by plotting the log concentration of complex 1 vs the inhibition percentage of cell viability and extrapolating the result of nonlinear regression using Prism 6 (Version 6.0b, GraphPad Software).

As stated hereinbefore that present invention investigated the cytotoxic effects of 1 in breast cancer cell line MCF-7 along with a standard reference drug oxaliplatin in both dose and time-dependent manner. The results from the MTT assay showed that complex 1 exhibit cytotoxic effects on MCF-7 cells after 24 h of incubation with IC50 values of 16.81 =1.33 μM whereas oxaliplatin inhibit the cell viability of MCF-7 cells with an IC₅₀ value of 31.4±0.69 μM after 24 h treatment. Interestingly, complex 1 showed cytotoxicity with IC₅₀ of 13.29±1.55 μM, whereas oxaliplatin exhibits 50% of toxicity at higher concentrations with IC₅₀ of 15.1±1.81 μM after 48 h, respectively (FIGS. 6a and 6b). The cell proliferation rate was observed under an inverted phase-contrast microscope after treatment of complex 1. The number of viable MCF-7 cells in the compound treated groups were significantly less than that of the untreated group (FIG. 6c).

The present study also investigated the cytotoxic effects of 1 in Human colon cancer cell lines HCT-116 and SW-480 on a dose dependent manner. XTT assay revealed that complex 1 exhibits cytotoxic effects on HCT-116 and SW-480 cells after 24 h of incubation with IC₅₀ values 15.27±1.18 μM and 10.04±1.98 M respectively. (FIG. 6.1)

In addition, results also showed that complex 1 did not impart any significant cytotoxicity in normal PBMCs and resulted in cell viabilities above 70% under the concentration range tested (5-60 μM). Therefore, it was evident from the above observations that IC₅₀ of complex 1 for PBMC must be >60 μM.

### Determination of Apoptosis and Morphological Changes Towards MCF-7 Cells and HCT-116 Cells via FACS and DAPI Fluorescence Study

FACS-based Annexin V-FITC/PI dual staining assay: MCF-7 cells were seeded in 6-well plate at 1×10⁵ cells per well and treated with an IC₅₀ dose of complex 1 and oxaliplatin for 24 h, after which they were harvested by trypsinization and processed into cell suspensions (10\hat{ }6 cells were taken from each group). Cells were centrifuged at 800 g before being re-suspended in a dye mix that included 250 μL Annexin V-FITC binding buffer, 1 μL PI solution, and 3 μL Annexin V-FITC solution as per assay kit protocol. For 15 minutes, the cell suspension was kept in dark. The BD FACS Verse was used to detect the intensities of red and green fluorescence, and Flowjo software (version 10) was used to assess the apoptotic rates of treated cells. Ten thousand events were collected to determine the population percentage. All experiments were repeated three times, and the results were presented as mean±SD.

DAPI staining by fluorescence microscopy: MCF-7 cells were seeded on sterile cover glasses, cultured for 24 h in a 6-well plate, and treated with IC₅₀ concentrations of complex 1. After 24 h of treatment, cells on the cover glass were fixed for 10 minutes with 4% paraformaldehyde and washed three times with 1×PBS. After that, cells were permeabilized with 0.1% Triton X-100 for 10 minutes before being rinsed three times with 1×PBS. The cells were then stained with DAPI staining solution (1 μg/mL) for 5 minutes while kept in the dark. Finally, a fluorescence microscope was used to take pictures of the cover glasses while they were mounted on the slides.

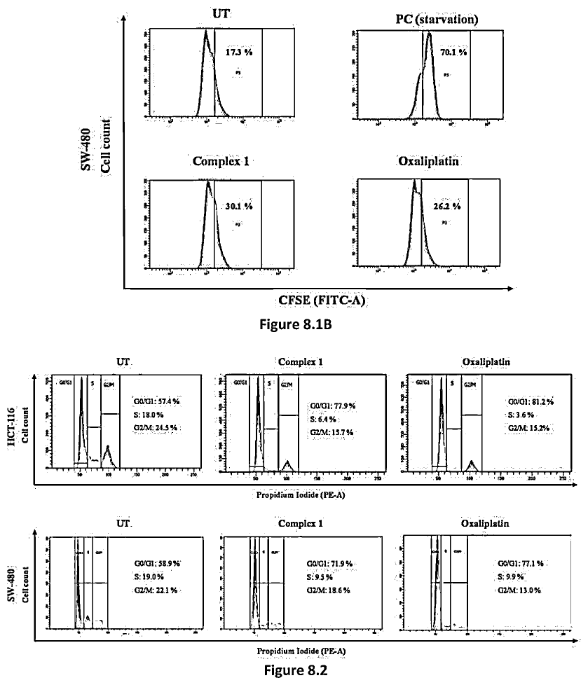

Flow cytometry analysis by Annexin-V FITC/PI-PE dual staining assay revealed that after 24 h treatment with the IC₅₀ dose of oxaliplatin and complex 1, the percentages of the late apoptotic (Annexin V+/PI+) population of MCF-7 cells were increased significantly from 2.87% to 23.9% and 20.1% respectively (***P<0.001). Similarly, the early apoptotic population (Annexin V+/PI−) was also significantly elevated in the treated groups (9.58% and 9.31% after treatment with oxaliplatin and complex 1 respectively) when compared to the untreated control group (4.96%) of cells. As a result, the complex significantly induces apoptosis to the same extent as that of the standard metallodrug oxaliplatin as there were no significant changes in the percentage value of apoptotic cell population between treatment with complex 1 and oxaliplatin. (NS, P>0.05) (FIGS. 7a and 7b). In addition, morphological alteration in MCF-7 cells were checked after treatment with the complex (at IC₅₀ concentration) for 24 h by DAPI staining. Under a fluorescent microscope, the metal complex-treated cancer cells showed signs of apoptosis including irregular chromatin condensation, fragmented nuclear structure and apoptotic bodies. (FIG. 7c).

Fluorescence microscopy using TUNEL staining in HCT-116 cells, showed increased number of TUNEL-positive cells and increased fluorescence intensity in complex 1 and oxaliplatin-treated groups in comparison to untreated control group (FIG. 7.1).

Colon Cancer cells: Flow cytometric analysis using Annexin V-FITC/PI-PE dual staining showed that complex 1 and oxaliplatin induced apoptosis in both cell lines. 24 h treatment with the respective IC₅₀ dose of complex 1 and oxaliplatin elevated the number of apoptotic cells compared to the untreated cells in both HCT-116 and SW-480 cell lines. The proportion of apoptotic cells was significantly higher in complex 1-treated group compared to oxaliplatin-treated group in both cell lines inferring complex 1 to be more promising than oxaliplatin (FIG. 7.2). Phase-contrast microscopy showed the presence of apoptotic bodies in complex 1- and oxaliplatin-treated groups (FIG. 7.3).

### Inhibition of Cell Proliferation and Cell Cycle Phase Arrest Study in MCF-7 Cells, and Cancer Cell Lines HCT-116 and SW-480 Through FACS

Cell proliferation and cycle study using FACS: To detect cell proliferation, CFSE dye was used. At first, MCF-7 cells were stained with 5 μM of CFSE dye for 15 min at 37° C. followed by the addition of FBS containing RPMI 1640 medium to end the reaction. Then CFSE-stained cells were cultured with both oxaliplatin and complex 1 for 24 h in 35-mm plates. One set of stained untreated cells was seeded only in complete media without any compound. For inhibition of proliferation, cells were grown in a serum-free medium (starvation) for 24 h as a positive control. On the next day, the green fluorescence intensity of CFSE within each cell was measured by flow cytometry, and data were calculated using Flowjo software (version 10). For cell cycle study, MCF-7 cells were seeded at 1×10⁵ cells per well in a 6-well plate, treated with an IC₅₀ dose of complex 1 and oxaliplatin for 24 h, then digested by trypsinization and made into cell suspension (10⁶ cells were harvested from each group) in 2% FBS containing 1×PBS, followed by fixation in 70% ethanol overnight at 4° C. After that cells were centrifuged at 850×g in a low-temperature centrifuge for 5 min. The ethanol solution was discarded and the cells were washed twice with 2% FBS containing 1×PBS. The cells were then treated for 30 minutes at 37° C. with 50 μl of RNase A (100 g/mL) and subsequently, 200 μl of PI (50 g/mL) was added. BD FACS flow cytometer was used to assess the difference in cell cycle phase distribution of nuclear DNA, which was then analyzed using Flowjo software (version 10). Ten thousand events were acquired to examine the population percentage. Both the cell proliferation and cell cycle experiments were repeated three times, and the data were provided as mean±SD.

Cancer cells exposed to chemotherapitic agents undergo cell cycle arrest at various stages in order to repair DNA damage and depending on the degree of cell damage, such cancer cells may either enter the process of cell division or die by apoptosis. Using flow cytometry after propidium iodide (PI) staining, we investigated whether the observed effect of complex 1 on cancer cell viability is due to induction of cell cycle arrest. MCF-7 cells exposed to the IC₅₀ concentration of 1 for 24 h showed an increased population of cells in the G2-M phase, 23.1%, compared to 8.88% in the untreated control group; however, the population of cells in the G0-G1 and S phases was also decreased, indicating cell cycle arrest in the G2-M phase. Furthermore, the oxaliplatin treatment increased the population of MCF-7 cells in the G2-M phase of the cell cycle to 21.3 percent (Figure. 8a and 8b). These results suggested that complex 1 arrested the cell cycle to a similar extent at the G2-M phase like oxaliplatin. Experiments using CFSE were conducted in order to directly assess cell proliferation. Following CFSE dye labeling, cells were given the IC₅₀ dose of both oxaliplatin and complex 1 for 24 h before FACS-based measurement of the remaining CFSE staining intensity. When CFSE enters inside the cells, it is transformed into a membrane-impermeable dye with a fluorescent tag that remains in the cytoplasm. The relative intensity of the dye decreases by half with each cycle of cell division as the retained CFSE is distributed equally among daughter cells. Importantly, the measured CFSE staining intensity after 24 h was significantly higher in both complex 1 and oxaliplatin treated cells compared to untreated controls in MCF-7 cells (IG. 8c and 8d). Cells were cultured for 24 h in media deprived of serum as a positive control for the suppression of proliferation. In between two-compounds, complex 1 inhibited proliferation almost as efficiently as oxaliplatin (FIG. 6c and 6d). These findings conclusively showed that complex 1 significantly reduced cell proliferation in MCF-7.

### Colon Cancer Cells (HCT-116 and SW-480)

Flow cytometric analysis of cell proliferation (using CFSE) and cell cycle distribution (using PI) showed that 24 h treatment with respective IC₅₀ dose of complex 1 and oxaliplatin suppressed the cell proliferation in both the cell lines. The antiproliferative efficacy of complex 1 was more than that of oxaliplatin in HCT-116 but was comparable to SW-480 (FIG. 8.1A and 8.1B). The population of cells in G0-G1 phase was increased with concomitant decrease in S and G2-M phase, indicating the cell cycle arrest at G0-G1 phase in both HCT-116 and SW-480 after 24 h treatment with complex 1 and oxaliplatin. In both HCT-116 and SW-480, the G0-G1 arrest was non-significant in complex 1-treated group and oxaliplatin-treated group. Therefore, complex 1 and oxaliplatin arrested cell cycle in a similar extent (FIG. 8.2).

Effects of complex 1 on cell cycle arrest and apoptotic signaling proteins in MCF-7 cells by western blot: Trans-Blot Turbo Transfer system was used to transfer the protein electrophoretically from gel to membrane and images were taken using the ChemiDoc XRS+ Gel Imaging System (Biorad).

To comprehend the signaling mechanism of complex 1 mediated cell cycle inhibitory activity, the protein expression of cell cycle inhibitors and other cell cycle-associated proteins was investigated. Western Blot technique was used to study the regulation of cell cycle and apoptosis-related proteins. MCF-7 cells were seeded at a concentration of 1×10⁶ cells per well in 6 well plates. After 24 h incubation, complex 1 was added for treatment. After 24 h, cells were washed with the ice-cold PBS and cell lysis was performed by cell scrapper by adding RIPA lysis solution and protease inhibitor cocktail. The cell lysate was then centrifuged for 15 minutes at 4° C. at 16,000 g. The supernatant was then collected and kept at 20° C., and the BCA Protein Assay Kit was used to assess protein concentrations. Equal amounts of proteins (30 g) were separated on a 12-15% SDS-PAGE gel for 1.30 h at 130V with a dual-color protein ladder. The resolved proteins were then electrophoretically transferred to a nitrocellulose membrane using the Trans-Blot Turbo Transfer system and the membranes were blocked for 1 hour with 5% BSA in Tris-buffered saline containing 0.1% Tween-20 (TBST). After blocking, the membranes were incubated with the respective primary antibody (1:1000 dilution) for an overnight period at 4° C. After that, membranes were washed three times with TBST for 10 mins at a time, and subsequently incubated for 1 hour at room temperature with HRP conjugated secondary antibody (1:2000 dilution). The blot was then washed three times with TBST before being developed with ECL substrate. The Biorad ChemiDoc XRS+ Gel Imaging System was used to capture images, and the data was analyzed using Biorad image lab 6.1 software.

Western blotting was used to evaluate the G2-M phase regulatory proteins Cyclin B1 and p-Cdc2^{Tyr15} in order to validate the outcomes of cell cycle analysis. For the present invention it is found that p-Cdc2^{Tyr15} (inactive form of Cdc2) expression was upregulated, whilst Cyclin B1 expression was shown to be downregulated (FIG. 9a and 9b) suggesting that it was blocking the entry of cells into the M phase. All the results suggested that complex 1 successfully promoted G2-M phase cell cycle arrest by preventing the development of the Cdc2/Cyclin B1 complex, by elevating the expression level of Wee1, p-Chk2^{Ser516} and p-CDC25C proteins (FIG. 9c and 9d). In the previous experiment using flow cytometry analysis it was observed that the complex 1 significantly triggered apoptosis and suppressed cell proliferation in MCF-7 cells. Therefore, the cell proliferation marker and apoptotic marker at the protein level were also assessed by western blotting to confirm the exact mechanism behind apoptosis. When comparing complex 1 treated MCF-7 cells to untreated cells, it was found that the levels of pro-apoptotic Bax protein were significantly enhanced, whereas the levels of antiapoptotic proteins Bcl-2 and Bel-xL were predominantly reduced (FIG. 9e and 9f). Furthermore, the level of tumor suppressive protein P53 was significantly upregulated, whereas cell proliferating biomarker PCNA was downregulated after treatment with complex 1 at IC₅₀ dose (FIG. 9e and 9f).

### Effects of Complex 1 on Cell Cycle Arrest and Apoptotic Signalling Proteins in Colon Cancer Cells

The western blot analysis was performed to delineate the underlying molecular mechanism of action for complex 1 upon both HCT-116 and SW-480 cell lines. The complex 1 downregulated the expression of Cyclin D, CDK4 and CDK6, thus arresting the cell cycle progression in G0-G1 phase (FIG. 9.1). The expression of pro-apoptotic proteins Bax and Bim were significantly upregulated and that of anti-apoptotic protein Bcl-xL was downregulated. The expression of executioner caspase-3 and cleaved PARP were also elevated which conferred the phenomenon of apoptosis (FIG. 9.2).

### Inhibition of Cell Migration of MCF-7 Cells by Complex 1

In vitro migration assay: To assess cell migratory potential, the scratch test (also known as the wound healing assay) was used. For this, MCF-7 cells were cultured in six-well plates for 24 h at a cell density of 1×10⁵ cells/mL in RPMI-1640 medium containing growth factors. The monolayer of cells was scraped with a disposable 200 μL plastic pipette tip in a streaking fashion. After that, cells from different groups (untreated, oxaliplatin treated and complex 1 treated) were cultured for up to 48 h in media, and cells were photographed to assess the area of the wound gap at various time intervals with an inverted microscope equipped with a digital camera. Images of the starting wound were compared to images at the end of the incubation period. All results are represented as mean±SD of three independent experiments.

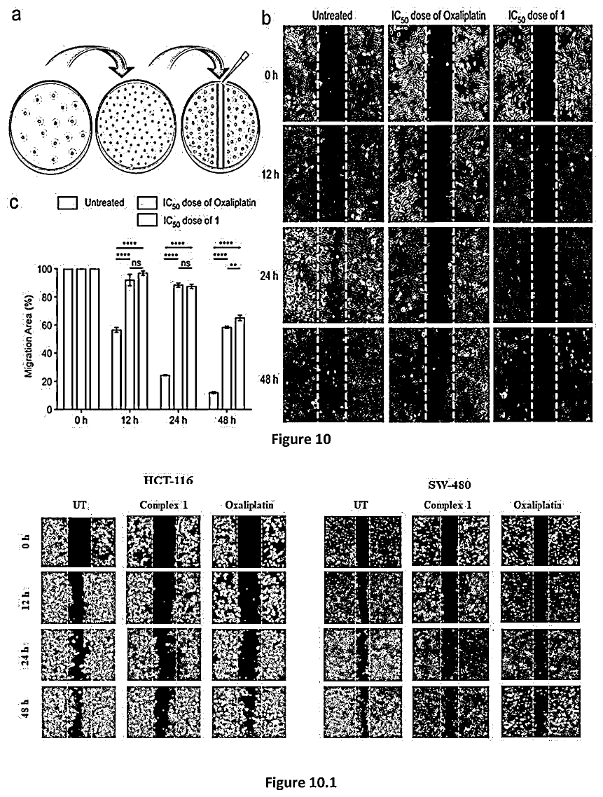

The wound area was under observation to evaluate the compound's inhibition capability for migration. The wound area reduced significantly in size in the untreated cells compared to treated cells. Cancer cells move from their originating site to a secondary site (metastatic ability) through a wound-healing property. But the cancer cells' wound does not heal following treatment with the present complex, and as a result, the cells lose their ability to migrate. Said cell migration rate of complex 1 is almost similar to oxaliplatin in magnitude (FIG. 10b and 10c).

### Inhibition of Cell Migration of Colon Cancer Cells by Complex 1

The wound healing assay (scratch assay) showed that treatment with IC₅₀ dose of complex 1 and oxaliplatin reduced the migration of cells compared to untreated cells in both HCT-116 and SW-480 in a time dependent manner. The wound area of complex 1-treated group and oxaliplatin-treated groups were statistically non-significant for all time points (FIG. 10.1).

Additionally to assess the role of different organic cation transporters (OCT1, OCT2, OCT3) in uptake of complex 1, a preliminary study was performed by using Dycenium-22 (a OCT3 inhibitor) in HCT-116 and SW-480. The MTT study revealed that in presence of the inhibitor, the IC₅₀ values of the complex 1 was increased showing decrease in its cytotoxic potency may be by reducing its uptake in cells. In the respective doses of inhibitor, the inhibitor was itself found to be non-toxic to the cells.

### Example-3

### Animal Studies

Experimental Animal: The mice used for the present invention were adult female Swiss Albino mice of approximately 20-25 g of weight, bred in an animal colony of the Chittaranjan National Cancer Institute, Kolkata. Central Experimental Animal Facility provided the mice with sterile paddy husk bedding encased in pathogen-free polypropylene cages with tight-fitting wire lids. The temperature and humidity were kept constant (23±2° C. and 55±10%) with alternate light and dark cycles (12 h/12 h). Animals were fed with a standard food pellet diet (EPIC rat and mice pellet from Kalyani Feed Milling Plant, Kalyani, West Bengal, India) and drinking water ad libitum. In each experiment, the mice were acclimatized for three days before being divided into distinct subgroups. All experimental procedures were firmly followed by the guidelines of the Institutional Animal Ethics committee (CPCSEA Reg. No. 1774/GO/RBi/S/14/CPCSEA, India).

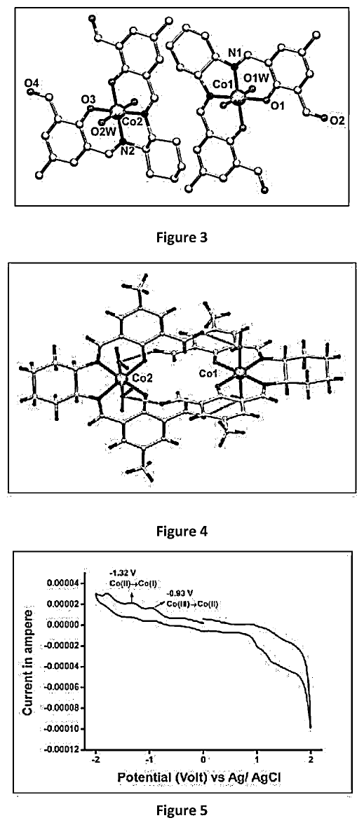

Experimental grouping for non-toxic dose determination: For the present invention, mice were randomized into five experimental groups (n=6) where one group was termed as the vehicle control group and an additional four as treatment groups (received 100 μL of consecutive doses of complex 1 intravenously) wherein Group 1 (vehicle control): Normal mice treated with 100 μL of 1×PBS, and for Group 2, Group 3, Group 4 and Group 5 normal mice treated with 10 μg/kg, 20 μg/kg, 30 μg/kg and 40 μg/kg body weight of complex 1 respectively. The whole experimental processes in determining the nontoxic dose are illustrated in (FIG. 2).

### Subacute Toxicity and Chronic Toxicity Study

For clinical relevance toxicity profiling is of utmost need for any new drug formulation. Both sub-acute toxicity and chronic toxicity of the metal complex 1 were evaluated. For histopathological analysis, tissue sections were cut using Leica microtome RM40, and photographs were taken by a brightfield microscope (Leica DM 1000). Hematological and biochemical parameters were analyzed by an automated hematology analyzer (KX-21, Sysmex, Japan) and clinical chemistry analyzer (AU400, Olympus, Tokyo, Japan) respectively.

To conduct subacute toxicity of complex 1, at first four experimental groups (Group 2 to Group 5) of normal female Swiss albino mice were administered with different doses of drugs for continuous 28 days whereas the vehicle control group (Group 1) received only PBS (FIG. 2). On the 29^{th} day, all experimental mice fasted for 4 h before euthanasia, and blood was collected with the help of a 22-gauge hypodermic needle by a retro-orbital puncture for evaluation of toxicological parameters and collected in two microcentrifuge tubes. Following that mice were sacrificed by euthanasia (overdose of Ketamine HCL, 160 mg/kg+Xylazine, 20 mg/kg i.p). The serum was collected from the blood sample after 30 minutes (post coagulation) for analyzing the biochemical parameters by centrifugation at 2000×g for 15 minutes and stored at −20° C. until analysis. The biochemical parameters of kidney and liver toxicity markers such as urea, creatinine, SGOT, SGPT, and ALP were evaluated from serum according to the manufacturer's protocol using an automated clinical chemistry analyzer. The blood sample for determining hematological parameters like RBCs, WBCs, HGB, and PLTs was collected in heparin-coated tubes and analyzed according to the manufacturer's protocol by an automated hematology analyzer. Chronic toxicity was also done similarly by administrating complex 1 for consecutive 40 days where all the biochemical parameters were analyzed by the automated clinical chemistry analyzer after sacrificing mice on the 41^{st} day (FIG. 2).

Subacute and chronic toxicity: Standardized sub-acute systemic toxicity study (28 days) specified that the animals at different dose groups (from 10 μg/kg of body weight to 40 μg/kg of body weight) did not show any significant changes in the haematological parameters like RBC, WBC, PLT, and HGB as compared to the untreated group of animals as seen in Table 2a. This implies that the novel cobalt complex 1 did not impart any significant haematological toxicity. Any alteration in the level of biochemical parameters such as SGOT, SGPT, ALP (indicative of drug-induced liver injury), or urea and creatinine (indicative of toxin-induced damaged renal function) express hepatotoxicity and nephrotoxicity however no such significant differences were noticed in those biochemical parameters as depicted in Table 2b. Therefore, no mortality or toxic symptoms were seen after 28 days of repetitive treatment (up to highest dosage i.e 40 μg/kg of body weight) in treated groups concerning the untreated group of animals. To see any adverse side effects of long-term exposure of complex 1, a standardized chronic toxicity study (40 days) was also carried out on normal Swiss albino mice. Data obtained from the experiment illustrated that both liver and kidney toxicity parameters remain within the normal range with select doses of complex 1 (10-40 μg/kg of body weight) for up to 40 days of continual treatment as mentioned in Table 3. Therefore, no chronic toxicity was observed concerning normal mice. These findings were further validated by histopathological evaluation.

TABLE 2a
Effect of complex 1 with different doses on hematological parameters of normal
female Swiss albino mice

| Groups | RBC(/μL) | WBC(/μL) | PLT(/μL) | HGB(g/DL) |
|---|---|---|---|---|
| Group 1 (Control) | 4.88 × 10⁶ (±0.9) | 14.8 × 10³ (±0.5) | 309 × 10³ (±8) | 9.3 (±0.5) |
| Group 2 (10 μg/kg) | 5.29 × 10⁶ (±0.9) | 15.3 × 10³ (±0.5) | 356 × 10³ (±6) | 8.0 (±1) |
| Group 3 (20 μg/kg) | 4.7 × 10⁶ (±0.6) | 15.8 × 10³ (±0.8) | 300 × 10³ (±19) | 8.7 (±0.4) |
| Group 4 (30 μg/kg) | 4.78 × 10⁶ (±0.6) | 14.3 × 10³ (±0.3) | 326 × 10³ (±12) | 8.5 (±0.65) |
| Group 5 (40 μg/kg) | 5.05 × 10⁶ (±0.5) | 17.2 × 10³ (±0.2) | 321 × 10³ (±8) | 9.0 (±0.2) |

The data are presented as mean ± SD of six independent samples from each group. After 28 days of successive administration with increasing doses of 1, there has been no significant difference in the modulation of hematological parameters (p >0.5).

TABLE 2b
Effect of complex 1 with different doses on biochemical parameters of
normal female Swiss albino mice

| | ALP | SGOT | SGPT | Urea | Creatinine |
|---|---|---|---|---|---|
| Groups | (IU/Lit) | (IU/Lit) | IU/Lit | (mg/dl) | (mg/dl) |
| Group 1 (Control) | 45.8 (±0.7) | 225 (±4) | 71 (±7) | 49 (±4) | 0.5 (±0.07) |
| Group 2 (10 μg/kg) | 55.3 (±3) | 229 (±9) | 85 (±6) | 47 (±6) | 0.4 (±0.06) |
| Group 3 (20 μg/kg) | 47.2 (±2) | 207 (±9) | 81 (±5) | 44 (±7) | 0.3 (±0.08) |
| Group 4 (30 μg/kg) | 48.3 (±2) | 206 (±4) | 76 (±6) | 43 (±3) | 0.6 (±0.06) |
| Group 5 (40 μg/kg) | 44.6 (±2) | 205 (±5) | 79 (±2) | 41 (±8) | 0.7 (±0.09) |

Values are represented as mean ± SD from six independent samples in each group. After 28 days of consecutive therapy with increasing doses of 1, there was no significant variation in liver and kidney toxicity markers (p >0.5).

TABLE 3
Modulation of liver and kidney toxicity parameters of normal
female Swiss albino mice for chronic toxicity evaluation

| | ALP | SGOT | SGPT | Urea | Creatinine |
|---|---|---|---|---|---|
| Groups | (IU/Lit) | IU/Lit) | IU/Lit | (mg/dl) | (mg/dl) |
| Group 1 (Control) | 47.8 (±2) | 220 (±6) | 81 (±7) | 49 (±7) | 0.6 (±0.07) |
| Group 2 (10 μg/kg) | 49.3 (±3) | 226 (±9) | 85 (±6) | 48 (±6) | 0.5 (±0.06) |
| Group 3 (20 μg/kg) | 47.6 (±4) | 225 (±3) | 86 (±5) | 47 (±5) | 0.4 (±0.04) |
| Group 4 (30 μg/kg) | 45.4 (±3) | 221 (±2) | 84 (±3) | 46 (±4) | 0.3 (±0.08) |
| Group 5 (40 μg/kg) | 46.8 (±7) | 227 (±4) | 82 (±2) | 49 (±9) | 0.2 (±0.09) |

Values are denoted as mean ± SD from six independent samples in each group. No significant changes were seen in liver and kidney toxicity parameters after 40 days of repeated treatment with increasing doses of 1 (p >0.5).

### Histopathological Evaluation of Complex 1 in the Normal Host System

Histopathological analysis: Liver and Kidney tissue samples were collected from mice of Group 1 (Vehicle control) and Group 5 (highest nontoxic dose) following euthanasia (Scheme 2). The samples from the particular experimental groups were immediately fixed in 10% neutral buffered formalin and the paraffin-embedded block was prepared following the standard procedure. After that, around 4 um thick paraffin tissue sections were cut using microtome RM40 and deparaffinized with Xylene followed by sequential alcohol rehydration and stained with hematoxylin and eosin (H&E). Additional special stains (PAS and Reticulin) were employed to identify the features of the kidney and liver tissue that are difficult to see on an H&E stain. The kit's manual was followed for staining with reticulin and PAS. Then, the photographs were taken from tissue section slides of different groups and examined under the bright field microscope.

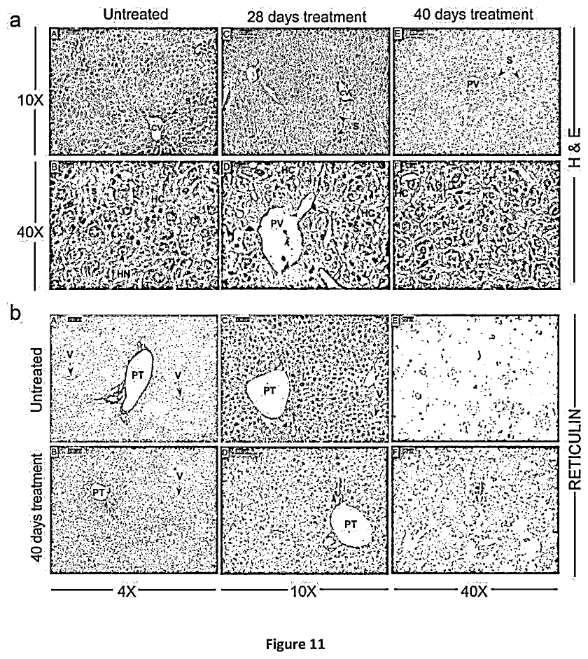

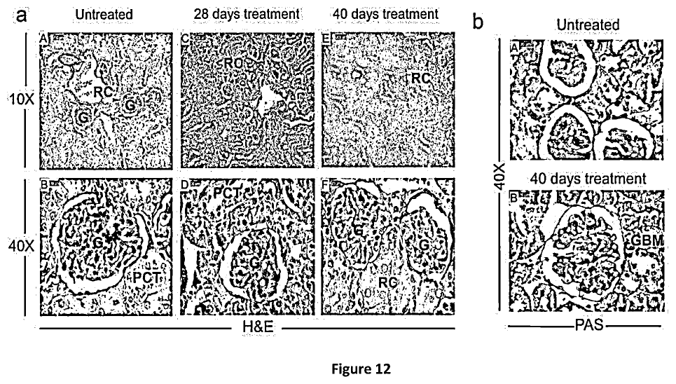

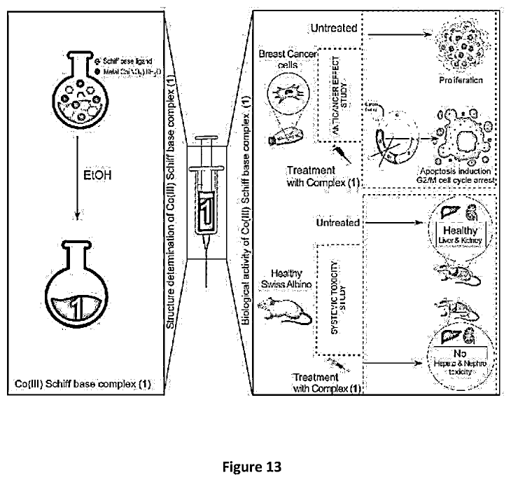

Histology of the kidney and liver of the normal host system (Swiss albino mice) was executed to check whether there is any disparity with the biochemical parameters. In comparison with the untreated group, no abnormalities in the histopathology of the kidney, and liver of the treated group of mice were detected, following 28 and 40 days of continuous treatment. Morphology of the liver of treated mice groups was similar to the untreated group as evident by H&E staining. The radial distribution pattern around the central vein (CV) was prominent with a smaller number of neutrophils followed by 28 days of uninterrupted treatment, indicating no ongoing acute inflammations are present. Radially arranged hepatocytes (HC) with an inadequate number of Kupffer cells (KC) in the liver tissue after 40 days of continuous treatment, implying lack of any chronic toxicity (FIG. 11a). Reticulin staining of liver of treated mice group (40 days) compared to untreated group revealed that the lining around the sinusoidal portal tract (PT) and hepatic venules (V) both were reticulines positive (as expected normally) but the intracellular spaces of the liver tissues were not, indicating the absence of any ongoing fibrotic changes (FIG. 11b). The renal corpuscles (RC) and glomerular tufts of kidney tissue sections did not exhibit any notable differences after 28 and 40 days of constant treatment when compared to the untreated group. Additionally, lack of glomerular infiltration, tubular dilation, and necrosis pointed towards the absence of continuous acute inflammation (FIG. 12a). PAS stain from untreated and treated mice group highlighted that there was no glomerular basement membrane (GBM) thickening and material deposition in mesangial kidney tissue after 40 days of treatment, indicating the absence of any chronic kidney damage (FIG. 12b).

Based on the current findings it can be concluded, that complex 1 induces apoptosis via G2-M cell cycle arrest in human breast cancer cell line MCF-7, and also induces apoptosis in colon cancer cell lines HCT-116 and/or SW-480 by arresting the cell cycle at the G0-G1 phase and show no significant hematological toxicity, nephrotoxicity, or hepatotoxicity in vivo. It offeres sufficient proof to establish itself as a promising chemotherapeutic candidate having strong anticancer effects without exhibiting significant toxicity to the host. Additionally, in vivo research on anticancer activity is needed to corroborate these results clinically. Active research is going on with this compound and we are anticipating a promising outcome from our laboratory in near future.

1. A non-toxic mononuclear side-off compartmental cobalt(III) Schiff base complex which is reaction product C₂₄H₃₀N₃O₁₀Co selectively of Co(III) containing Schiff base ligand and derived of 1,2-diaminocyclohexane (DACH) and 2,6-diformyl-4-methylphenol (Dif) which is capable of exhibiting cytotoxicity at lower concentration against cancerous cells for 24 h of therapy without being overly toxic to the normal healthy human cell line (PMBC).

2. The non-toxic mononuclear side-off compartmental cobalt(III) Schiff base complex as claimed in claim 1, which is reaction product C₂₄H₃₀N₃O₁₀Co selectively of Co(III) containing Schiff base ligand and derived of 1,2-diaminocyclohexane (DACH) and 2,6-diformyl-4-methylphenol (Dif) which is capable of causing apoptosis in breast cancer cell line MCF-7 cells by arresting the cell cycle at the G2-M phase at much lower concentrations than oxaliplatin having IC₅₀ values of 16.81±1.33 μM in compare to IC₅₀ value of 31.4±0.69 μM after 24 h treatment against MCF-7 cells without being overly toxic to healthy host systems.

3. The non-toxic mononuclear side-off compartmental cobalt(III) Schiff base complex as claimed in claim 1, which is reaction product C₂₄H₃₀N₃O₁₀Co selectively of Co(III) containing Schiff base ligand and derived of 1,2-diaminocyclohexane (DACH) and 2,6-diformyl-4-methylphenol (Dif) which is capable of causing apoptosis in colon cancer cell lines HCT-116 and SW-480 by arresting the cell cycle at the G0-G1 phase having IC₅₀ values 15.27±1.18 μM and 10.04±1.98 μM respectively comparable with the IC₅₀ values of oxaliplatin which are 16.73±1.78 μM and 7.87±1.54 μM respectively for HCT-116 and SW-480 cells after 24 h treatment.

4. The non-toxic mononuclear Schiff base complex as claimed in claim 1, having-fluorescence property, displays a broad band consisting of two small breaks at 1634 cm⁻¹ and 1642 cm⁻¹ and sharp bands at ˜1550 cm⁻¹ and ˜1385 cm⁻¹ by FTIR,
shows a strong band at λₘₐₓ˜410 nm in methanol, but two bands at λₘₐₓ˜412 nm and 354 nm in 0.03% DMSO-H₂O solution and stable upto one week in 0.03% DMSO-H₂O medium as indicated by UV-Vis spectroscopy;
retention time of 2.018 min in HPLC run on an C-18, Reserve Phase Column from WATERS (4.6×150 mm, particle size 5 μm), using 0.1% TFA in H₂O and 100% CH₃CN as mobile phase;
retains its mononuclear entity in the solution phase and presence of cobalt ion in the +3 oxidation state is confirmed by ESI-MS and Cyclic Voltammetry study respectively; and fluoresces well under microscope after entering the cells.

5. The non-toxic mononuclear Schiff base complex as claimed in claim 1, exhibit cytotoxic effects on MCF-7 cells after 24 h of incubation with IC₅₀ values of 16.81±1.33 μM whereas oxaliplatin inhibit the cell viability of MCF-7 cells with an IC₅₀ value of 31.4±0.69 μM after 24 h treatment causing significant reduction of the number of viable MCF-7 cells than that of the untreated group whereas do not impart any significant cytotoxicity in normal PBMCs and resulted in cell viabilities above 70% under the concentration range tested (5 μM-60 μM) i.e. IC₅₀ of said Schiff base complex for PBMC is >60 μM.

6. The non-toxic mononuclear Schiff base complex as claimed in claim 1, wherein the cobalt(III) Schiff base complex which is reaction product C₂₄H₃₀N₃O₁₀Co synergistically triggers apoptosis and suppressed cell proliferation in MCF-7 cells wherein the levels of pro-apoptotic Bax protein and tumor suppressive protein P53 are significantly enhanced, and the levels of antiapoptotic proteins Bcl-2 and Bcl-xL and cell proliferating biomarker PCNA are significantly downregulated and displays the cell migration rate almost similar to oxaliplatin in magnitude after treated with said Schiff base complex at IC₅₀ dose.

7. The non-toxic mononuclear Schiff base complex as claimed in claim 1, wherein cobalt(III) Schiff base complex which is reaction product C₂₄H₃₀N₃O₁₀Co which upon exposure to MCF-7 cells at the IC₅₀ concentration of said Schiff base complex for 24 h showed an increased population of cells in the G2-M phase, 23.1%, compared to 8.88% in the untreated control group; along with reduction of the population of cells in the G0-G1 and S phases indicating cell cycle arrest in the G2-M phase wherein the oxaliplatin treatment increased the population of MCF-7 cells in the G2-M phase of the cell cycle to 21.3% indicating a similar extent of the cell cycle arrest at the G2-M phase like oxaliplatin.

8. The non-toxic mononuclear Schiff base complex as claimed in claim 1, wherein cobalt(III) Schiff base complex which is reaction product C₂₄H₃₀N₃O₁₀Co upon standardized sub-acute systemic toxicity study (28 days) and/or standardized chronic toxicity study (40 days) specified that the animals at different dose groups (from 10 μg/kg of body weight to 40 μg/kg of body weight) do not show any significant changes in the haematological parameters like RBC, WBC, PLT, and HGB as compared to the untreated group of animals implying no significant haematological toxicity and any alteration in the level of biochemical parameters such as SGOT, SGPT, ALP (indicative of drug-induced liver injury), or urea and creatinine (indicative of toxin-induced damaged renal function) expressing hepatotoxicity and nephrotoxicity are not noticed in those biochemical parameters.

9. A process for the preparation of the non-toxic mononuclear Schiff base complex as claimed in claim 1, comprising the steps of:
providing ethanolic solution of 2,6-diformyl-4-methylphenol;
providing adding ethanolic solution of (±) cyclohexane-1,2-diamine with stirring;
adding Co(NO₃)₂·6H₂O in situ followed by refluxing to yield the non-toxic mononuclear side-off compartmental cobalt(III) Schiff base complex C₂₄H₃₀N₃O₁₀Co.

10. The process for preparation of the Schiff base complex as claimed in claim 9, comprising the steps of:
providing ethanolic solution 5 ml to 10 ml preferably 5 ml of 2,6-diformyl-4-methylphenol 0.162 g (0.9878 mmol) to 0.166 g (1.0122 mmol) preferably 0.164 g, (1 mmol);
adding dropwise ethanolic solution of (±) cyclohexane-1,2-diamine 0.055 g (0.4824 mmol) to 0.0059 g (0.5175 mmol) preferably 0.057 g (0.5 mmol) with stirring;
adding Co(NO₃)₂·6H₂O 0.289 g (0.9931 mmol) to 0.293 g (1.0069 mmol) preferably 0.291 g, (1 mmol) in situ followed by refluxing for (22 h to 26 h preferably 24 h at 100° C., providing a dark brown-colored solution which was filtered and solvent evaporated off; and
washing the residual solid several times with ice cold water and then solubilized in methanol which upon keeping in desiccator provide dark brown crystals of the Schiff base complex.
